(19) United States
(12) Patent Application Publication
(10) Pub. No.: US 2020/0140413 A1
(43) Pub. Date: May 7, 2020

(54) WATER-SOLUBLE TRIMETHOXYPHENYLPYRIDINE-TYPE COMPLEXING AGENTS, AND CORRESPONDING LANTHANIDE COMPLEXES

(71) Applicant: CISBIO BIOASSAYS, Codolet (FR)

(72) Inventors: Laurent LAMARQUE, SAINT-VICTOR LA COSTE (FR); Jurriaan ZWIER, ROCHEFORT-DU-GARD (FR); Emmanuel BOURRIER, BAGNOLS-SUR-CEZE (FR)

(21) Appl. No.: 16/622,452

(22) PCT Filed: Jun. 13, 2018
(86) PCT No.: PCT/FR2018/051392

§ 371 Date: Dec. 13, 2019

(30) Foreign Application Priority Data
Jun. 14, 2017 (FR) 1755330

(51) Int. Cl. C07D 401/14; C07F 5/00; G01N 33/52

(52) U.S. Cl. CPC C07D 401/14; G01N 33/52; C07F 5/003

(57) ABSTRACT

The invention relates to complexing agents of formula (I):

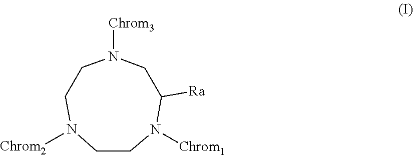

in which Ra, Chrom₁, Chrom₂ and Chrom₃ are as defined in the description. The invention also relates to lanthanide complexes obtained from these complexing agents. The invention can be used for labelling biological molecules.

The present invention relates to water-soluble complexing agents or ligands, lanthanide complexes obtained from these complexing agents, and to the use of these lanthanide complexes for labelling molecules and detecting them by time-resolved fluorescence techniques. This invention describes stable complexes comprising one, two or three water-soluble functionalized trimethoxyphenylpyridine-type chromophores.

### PRIOR ART

The use of lanthanide complexes has increased considerably over about the last twenty years in the field of life sciences. These fluorescent compounds in fact have interesting spectroscopic characteristics, which make them markers of choice for detecting biological molecules. These fluorescent compounds are particularly suitable for use in conjunction with compatible fluorophores for performing FRET (Förster Resonance Energy Transfer) measurements, application of which for studying the interactions between biomolecules is exploited commercially by several companies, including Cisbio Bioassays and its HTRF® product range. The relatively long lifetime of the lanthanide complexes also makes it possible to perform time-resolved fluorescence measurements, i.e. with a delay after excitation of the fluorophores, which makes it possible to limit the fluorescence interferences due to the measurement medium. The latter characteristic is all the more useful as the measurement medium becomes closer to a biological medium that comprises many proteins whose fluorescence could interfere with that of the compounds being studied.

Several lanthanide complexes have been disclosed and some are exploited commercially: mention can be made in particular of macropolycyclic lanthanide cryptates (EP-A-0 180 492, EP-A-0 321 353, EP-A-0 601 113, WO 2001/96877, WO 2008/063721), lanthanide complexes comprising a unit derived from coumarin bound to a diethylenetriamine penta-acid unit (U.S. Pat. No. 5,622,821), and those comprising derivatives of pyridine (U.S. Pat. Nos. 4,920,195, 4,761,481), of bipyridine (U.S. Pat. No. 5,216,134), or of terpyridine (U.S. Pat. Nos. 4,859,777, 5,202,423, 5,324,825).

The fluorescent lanthanide complexes consist of three parts:
a light-absorbing chromophore (antenna effect),
a complexing part
and an atom belonging to the lanthanide group (generally europium or terbium).

A great many chromophores have been used by the teams working in this field, and their work has been the subject of many review articles: Journal of Luminescence 1997, 75, 149; Chemical Reviews 2010, 110, 2729; and Inorganic Chemistry 2014, 53, 1854. Among all these works, few of them deal with the trimethoxyphenylpyridine chromophore. In Journal of Luminescence 1997, 75, 149, the authors describe the photophysical properties of derivatives of trimethoxyphenyldipicolinic acid, which is formally a chelate that is unstable in aqueous environments. In Analytical Chemistry 2005, 77, 2643, the authors introduced this unit into nanoparticles in order to make them usable in an immunoassay. However, these particles are large (45 nm in diameter), which is a drawback when small biological molecules are to be labeled with fluorescent probes.

Application WO 89/04826 relates to the synthesis of lanthanide complexes comprising three trimethoxyphenylpyridine-type chromophores. These complexes belong to the group of chelates, which makes them very unstable complexes especially in the presence of divalent cations or complexing agents of the EDTA type, which are used as additives in immunoassay buffers.

In application WO 2005/058877, the authors describe complexes comprising trimethoxyphenylpyridine units. These chromophores have been incorporated in various structures:
when they are incorporated in chelates, they form complexes that are unstable in the abovementioned media (divalent cations and EDTA);
when the chromophores are integrated in macrocycles of the triazacyclononane type (TACN), this time the complexes are stable but have quite low solubility in biological aqueous media. Moreover, functionalization is not possible directly, and the authors do not describe any procedure for functionalizing these systems;
finally, when these chromophores are incorporated in macrocycles of the triazacyclodecane type, the complexes also still have quite low solubility in the biological aqueous media. Functionalization is carried out on the middle carbon of the propylene chain of the macrocycle, which alters the symmetry about the lanthanide atom and consequently the distribution of the lines of the emission spectrum.

The invention aims to overcome the drawbacks of the compounds of the prior art by supplying complexes that are stable in the presence of EDTA and with most divalent cations, soluble in all biological media since the complexes of the invention comprise hydrosolubilizing groups of the anionic, cationic or zwitterionic type and finally a functionalization arm directly substituted on the ethylene chain of the triazacyclononane ring, a ring that is particularly suitable for complexation of the lanthanide atom and which obeys type C3 symmetry around the lanthanide. The complexes of the invention supply compounds whose emission spectrum is well suited to their use in FRET experiments, as well as being very convenient for labeling biomolecules.

### BRIEF DESCRIPTION OF THE FIGURES

FIGS. 1 to 3 show respectively the UV spectrum, the chromatogram and the mass spectrum of a representative complex of the invention.

FIGS. 4 to 6 show respectively the UV spectrum, the chromatogram and the mass spectrum of a representative complex of the invention.

FIGS. 7 to 9 show respectively the UV spectrum, the chromatogram and the mass spectrum of a representative complex of the invention.

### COMPLEXING AGENTS

The complexing agents according to the invention are the compounds of formula (I):

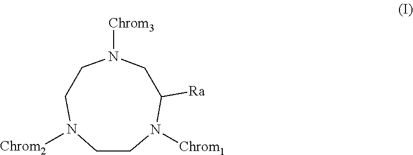

in which:
Chrom₁, Chrom₂ and Chrom₃ each represent a group of formula (Ia) or (Ib):

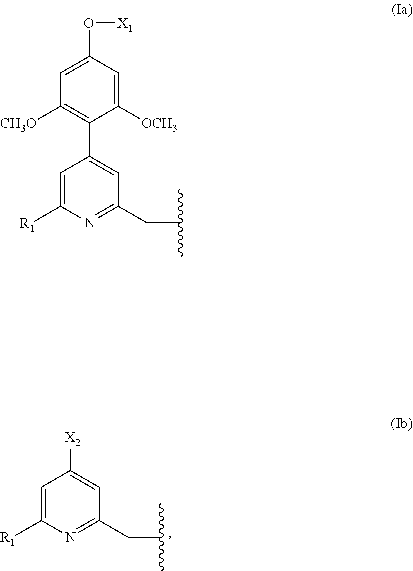

X₁ and X₂ each represent a group L₁-CO—R or L₂-G;
R is a group —OR₂ or —NH-E;
Ra is H or a group —(CH₂)ₗ-G;
R₁ is a group —CO₂H or —PO(OH)R₃,
R₂ is H or a (C₁-C₄)alkyl;
R₃ is a (C₁-C₄)alkyl, preferably a methyl; a phenyl optionally substituted with a group —SO₃⁻, the latter preferably being in the meta or para position; or a benzyl;
L₁ is a direct bond; a group —(CH₂)ᵣ— optionally interrupted by at least one atom selected from an oxygen atom, a nitrogen atom and a sulfur atom; a —CH═CH— group; a —CH═CH—CH₂— group; a —CH₂—CH═CH— group; or a PEG group;
L₂ is a divalent linking group;
G is a reactive group;
E is a group —CH₂—(CH₂)ₛ—CH₂—SO₃⁻ or —N⁺Alk₁Alk₂Alk₃, or a sulfobetaine;
l is an integer in the range from 1 to 4;
r is an integer in the range from 1 to 6, preferably from 1 to 3;
s is 0, 1 or 2;
Alk₁, Alk₂, Alk₃, which may be identical or different, represent a (C₁-C₆)alkyl;
   it being understood that the compound of formula (I) comprises at least one group of formula (Ia) and at least one group L₁-CO—R.

“PEG group” means a polyethylene glycol group of formula —CH₂—(CH₂OCH₂)_{y}—CH₂OCH₃, y being an integer in the range from 1 to 5.

“Sulfobetaine” means a group selected from:

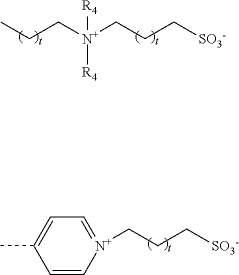

with R₄ representing a (C₁-C₆)alkyl, preferably a methyl or ethyl, and t being equal to 1, 2, 3, 4, 5 or 6, and preferably equal to 1 or 2, the sulfobetaine of formula —(CH₃)₂N⁺—(CH₂)₃—SO₃⁻ being preferred.

The groups —SO₃H, —CO₂H and —PO(OH)₂ are in deprotonated or non-deprotonated form, depending on the pH. These groups therefore also denote hereinafter the groups —SO₃⁻, —CO₂⁻ and —PO(OH)O⁻, and vice versa.

A first preferred family of complexing agents consists of the compounds of formula (I) where Chrom₁ represents a group of formula (Ia) in which X₁ is a group L₂-G; and Chrom₂ and Chrom₃, which may be identical or different, each represent a group of formula (Ib) in which X₂ is a group L₁-CO—R. In one embodiment, Chrom₂ and Chrom₃ are identical.

A second preferred family of complexing agents consists of the compounds of formula (I) where Chrom₁ and Chrom₂, which may be identical or different, each represent a group of formula (Ia) in which X₁ is a group L₁-CO—R; and Chrom₃ represents a group of formula (Ib) in which X₂ is a group L₂-G. In one embodiment, Chrom₁ and Chrom₂ are identical.

In one embodiment common to the first two preferred families of complexing agents, Ra is H.

A third preferred family of complexing agents consists of the compounds of formula (I) where Chrom₁, Chrom₂ and Chrom₃, which may be identical or different, each represent a group of formula (Ia) in which X₁ is a group L₁-CO—R; and Ra is a group —(CH₂)ₗ-G. In one embodiment, Chrom₁, Chrom₂ and Chrom₃ are identical.

Among these three preferred families, preferred subfamilies are those where the complexing agents comprise one or more of the following characteristics:
R₁ is a group —CO₂H or —P(O)(OH)R₃ in which R₃ is a (C₁-C₄)alkyl or a phenyl;
L₁ is a direct bond; a group —(CH₂)ᵣ— optionally interrupted by at least one atom selected from an oxygen atom and a sulfur atom, and r=2 or 3; a —CH═CH— group; a —CH═CH—CH₂— group; or a —CH₂—CH═CH— group;
E is a group —CH₂—(CH₂)ₛ—CH₂—SO₃⁻ with s=0 or 1; —(CH₂)ₛ—N⁺Alk₁Alk₂Alk₃ with Alk₁, Alk₂ Alk₃, which may be identical or different, representing a (C₁-C₄)alkyl and s=0 or 1; or a group of formula:

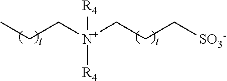

in which R₄ is a (C₁-C₄)alkyl and t is 1 or 2.

In one embodiment of the invention, when the complexing agents of formula (I) comprise several groups E, at most one of these groups represents a sulfobetaine.

The reactive group G carried by a spacer arm L₂ makes it possible to couple the compounds according to the invention to a species that is wished to make fluorescent, for example an organic molecule, a peptide, a protein or a nucleotide (RNA, DNA). The techniques for conjugation of two organic molecules are based on the use of reactive groups and form part of the general knowledge of a person skilled in the art. These conventional techniques are described for example in Bioconjugate Techniques, G. T. Hermanson, Academic Press, Second Edition 2008, p. 169-211.

Typically, the reactive group is an electrophilic or nucleophilic group that can form a covalent bond when it is brought into the presence of a suitable nucleophilic or electrophilic group, respectively. The reaction of conjugation between a compound according to the invention comprising a reactive group and an organic molecule, a peptide or a protein bearing a functional group leads to the formation of a covalent bond comprising one or more atoms of the reactive group.

Preferably, the reactive group G is a group derived from one of the following compounds: an acrylamide, an activated amine (for example a cadaverine or an ethylenediamine), an activated ester, an aldehyde, an alkyl halide, an anhydride, an aniline, an azide, an aziridine, a carboxylic acid, a diazoalkane, a haloacetamide, a halotriazine, such as monochlorotriazine, dichlorotriazine, a hydrazine (including the hydrazides), an imido ester, an isocyanate, an isothiocyanate, a maleimide, a sulfonyl halide, or a thiol, a ketone, an amine, an acid halide, a succinimidyl ester, a hydroxysuccinimidyl ester, a hydroxysulfosuccinimidyl ester, an azidonitrophenyl, an azidophenyl, a 3-(2-pyridyldithio)propionamide, a glyoxal, a triazine, an acetylene group, and in particular a group selected from the groups of formulae:

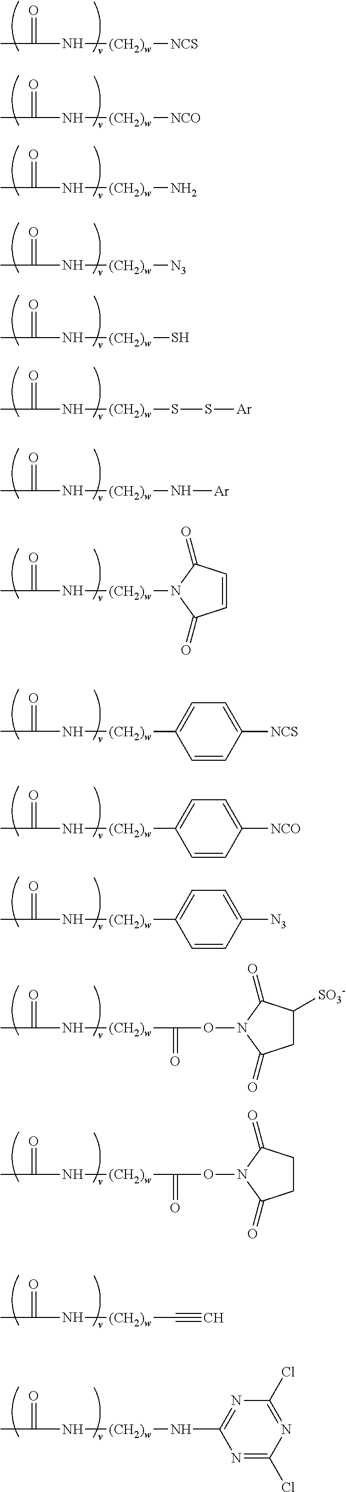

in which w varies from 0 to 8 and v is equal to 0 or 1, and Ar is a saturated or unsaturated 5- or 6-membered heterocycle, comprising 1 to 3 heteroatoms, optionally substituted with a halogen atom.

Preferably, the reactive group G is an amine (optionally protected in the form —NHBoc), a succinimidyl ester, a haloacetamide, a hydrazine, an isothiocyanate, a maleimide group, or a carboxylic acid (optionally protected in the form of a group —CO₂Me, —CO₂tBu). In the latter case, the acid will have to be activated in the form of ester so as to be able to react with a nucleophilic species.

The reactive groups G are bound to the complexing agent by a covalent bond or else via a spacer arm advantageously consisting of a divalent organic radical. Thus, the spacer arm L₂ may be selected from:
a direct bond;
a linear or branched C₁-C₂₀ alkylene group, optionally containing one or more double or triple bonds;
a C₅-C₈ cycloalkylene group; a C₆-C₁₄ arylene group;
   said alkylene, cycloalkylene or arylene groups optionally containing one or more heteroatoms, such as oxygen, nitrogen, sulfur, phosphorus or one or more carbamoyl or carboxamido group(s), and said alkylene, cycloalkylene or arylene groups optionally being substituted with 1 to 5, preferably 1 to 3, C₁-C₈ alkyl, C₆-C₁₄ aryl, sulfonate or oxo groups;
a group selected from the divalent groups of the following formulas:

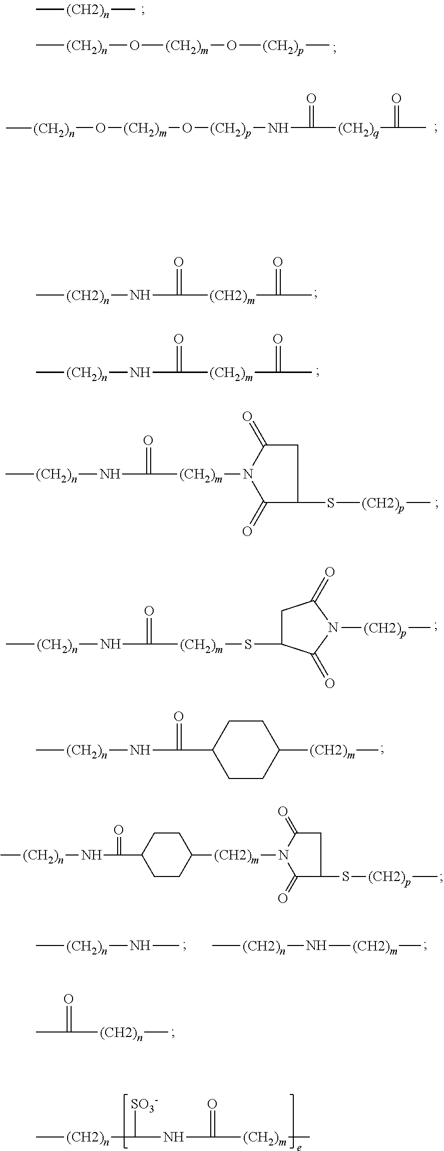

in which n, m, p, q are integers from 1 to 16, preferably from 1 to 5 and e is an integer in the range from 1 to 6, preferably from 1 to 4.

Preferably, the group -L₂-G consists of a reactive group G selected from: a carboxylic acid (optionally protected in the form of a group —CO₂Me, —CO₂tBu), an amine (optionally protected in the form —NHBoc), a succinimidyl ester, a haloacetamide, a hydrazine, an isothiocyanate, a maleimide group, and a spacer arm L₂ consisting of an alkylene chain comprising from 1 to 5 carbon atoms or a group selected from the groups of formula:

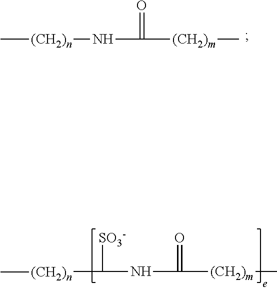

where n and m are integers from 1 to 16, preferably from 1 to 5 and e is an integer in the range from 1 to 6, preferably from 1 to 4, the group G being bound to one or other end of these divalent groups.

### DESCRIPTION OF THE INVENTION

The abovementioned problems were resolved by means of complexing agents consisting of a triazotized macrocycle (1,4,7-triazacyclononane, TACN hereinafter), the nitrogen atoms of which are substituted with trimethoxyphenylpyridine-type chromophores in which the methoxy in position 4 has been replaced with a group O—X₁, which allows hydrosolubilizing functions to be introduced easily. The complexing agents according to the invention form stable complexes with the lanthanides, and may be used for producing fluorescent conjugates of molecules of interest. The lanthanide complexes according to the invention have excellent photophysical properties, in particular in respect of their quantum efficiency, luminescence lifetime and excitation spectrum, which is very suitable for laser excitation at about 337 nm. The complexes of the invention may comprise one, two or three chromophores, which allows easy modulation of the overall brightness of the complex as well as the size of the complex. When the complex comprises a chromophore there is little steric hindrance. The presence of three chromophores significantly increases the coefficient of molar absorption (epsilon) and consequently the overall brightness of the complex, and the solubility of the complexes in an aqueous medium makes them very suitable for use in biological media. Finally the NH₂ function carried by the TACN ring allows easy bioconjugation with biomolecules. In particular, this function is easily convertible to N-hydroxysuccinimide ester, the biologists' preferred function.

### Complexes

The invention also relates to the lanthanide complexes consisting of a lanthanide atom complexed by a complexing agent as described above, the lanthanide being selected from: Eu^{3+,} Sm^{3+,} Tb^{3+,} Gd³⁺, Dy³⁺, Nd³⁺, Er³⁺. Preferably, the lanthanide is Tb³⁺, Sm³⁺ or Eu³⁺ and even more preferably Tb³⁺.

These complexes are prepared by bringing into contact the complexing agents according to the invention and a lanthanide salt. Thus, reaction between one equivalent of complexing agent and 1 to 5 equivalents of lanthanide salt (europium, samarium or terbium in the form of chlorides, acetates or triflates) in a solvent (acetonitrile, methanol or other solvent compatible with these salts) or a buffer, at room temperature for some minutes, leads to the corresponding complex.

As pointed out above, the fluorescent complexes obtained have excellent photophysical properties, in particular in respect of their quantum efficiency, luminescence lifetime and their excitation spectrum, which is very suitable for laser excitation at about 337 nm. Moreover, the distribution of the bands of their emission spectra endows the complexes with very favourable properties in a FRET application with acceptors of the cyanine, fluorescein, rhodamine or allophycocyanin type (such as XL665 marketed by Cisbio Bioassays). Owing to the great stability of these complexes in biological media containing most of the divalent cations (Ca²⁺, Mg²⁺ . . . ) or EDTA, their luminescence remains excellent compared to the complexes of the prior art.

### Conjugates

The complexing agents and lanthanide complexes according to the invention comprising a group -L₂-G are particularly suitable for labelling organic or biological molecules comprising a functional group capable of reacting with the reactive group to form a covalent bond. Thus, the invention also relates to the use of the lanthanide complexes for labelling molecules of interest (proteins, antibodies, enzymes, hormones, RNA, DNA etc.).

The invention also relates to the molecules labelled with a complex according to the invention. All the organic or biological molecules can be conjugated with a complex according to the invention if they possess a functional group capable of reacting with the reactive group. In particular, the conjugates according to the invention comprise a complex according to the invention and a molecule selected from: an amino acid, a peptide, a protein, an antibody, a sugar, a carbohydrate chain, a nucleoside, a nucleotide (DNA, RNA), an oligonucleotide, an enzyme substrate (in particular a suicide enzyme substrate such as a benzylguanine or a benzylcytosine (enzyme substrates marketed under the names Snaptag and Cliptag)), a chloroalkane (enzyme substrate marketed under the name Halotag), coenzyme A (enzyme substrate marketed under the name ACPtag or MCPtag).

### Synthesis

The general strategy regarding preparation of the complexing agents (ligands) and the complexes according to the invention is described schematically below (scheme 1: mono-antenna, scheme 2: di-antenna and scheme 3 tri-antenna), and in more detail in the experimental section.

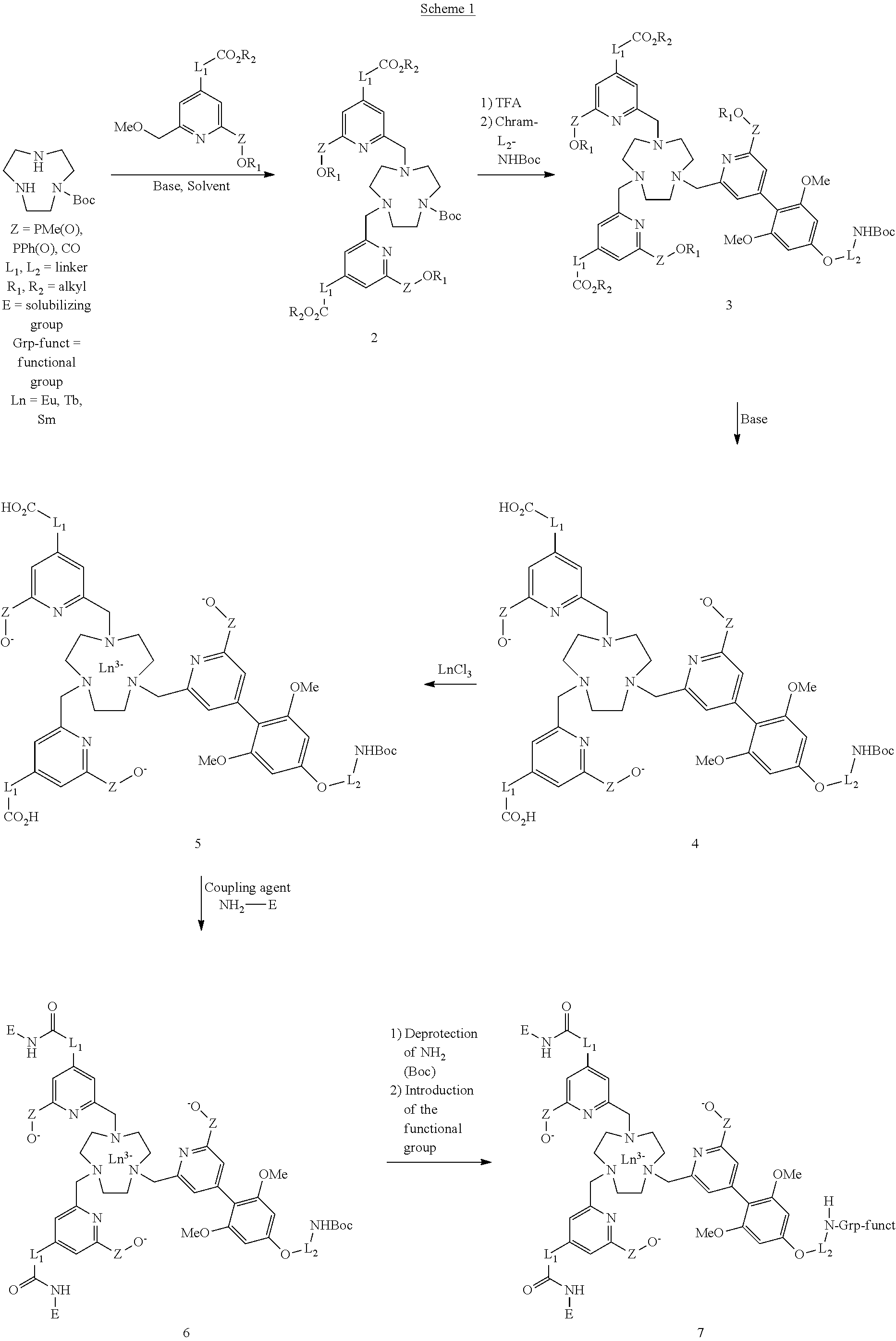

Starting from the monoprotected triazacyclononane macrocycle Boc 1, the two pyridinyl units were introduced which will be used for attaching the two solubilizing groups E. The protective group Boc is removed and then the antenna (chromophore) is added onto the macrocycle, thus leading to the ligand 3. Hydrolysis of the esters (carboxylates and phosphinates) was carried out conventionally using basic conditions. This then makes it possible to incorporate the lanthanide atom (Ln), thus forming the complexes 5. Starting from these complexes, the two hydrosolubilizing functions E were introduced. Finally, after deprotection of the protective group Boc carried by the antenna (chromophore), the complexes were functionalized (7) so that they can be conjugated on biomolecules.

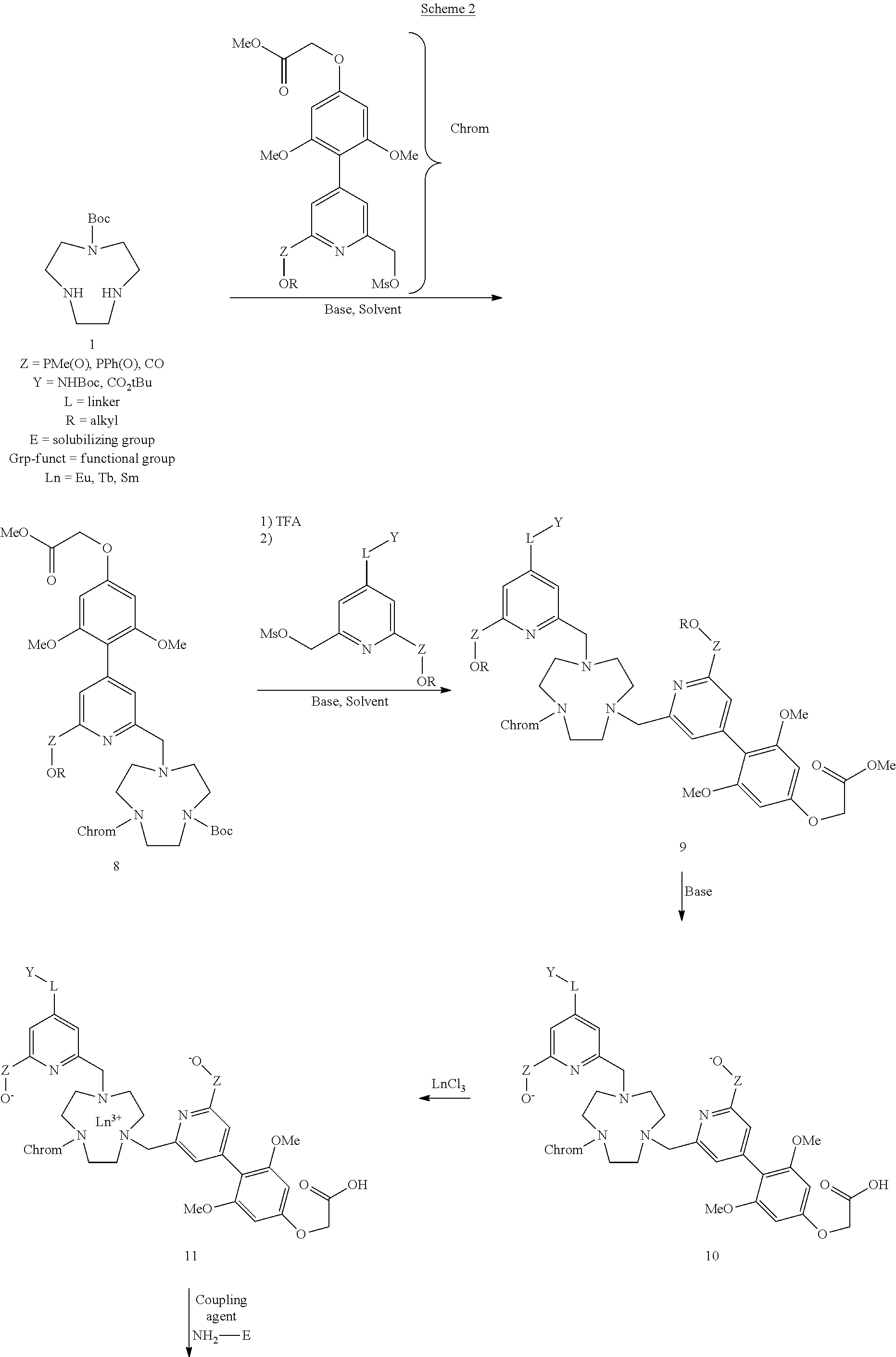

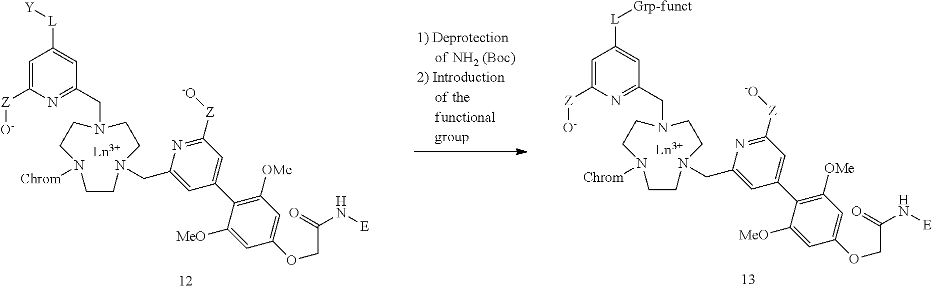

The di-antenna systems are obtained using a similar strategy but reversing the order of introducing the pyridinyl units and chromophores. This time the antennas are introduced first, leading to the compounds 8. After removing the Boc group, the last pyridinyl unit was introduced. The rest is identical, namely hydrolysis of the ester functions (carboxylates and phosphinates), formation of the lanthanide complex, introduction of the two hydrosolubilizing functions E (this time these functions are carried by the chromophores) and then incorporation of the functional group, thus leading to the di-antenna family 13.

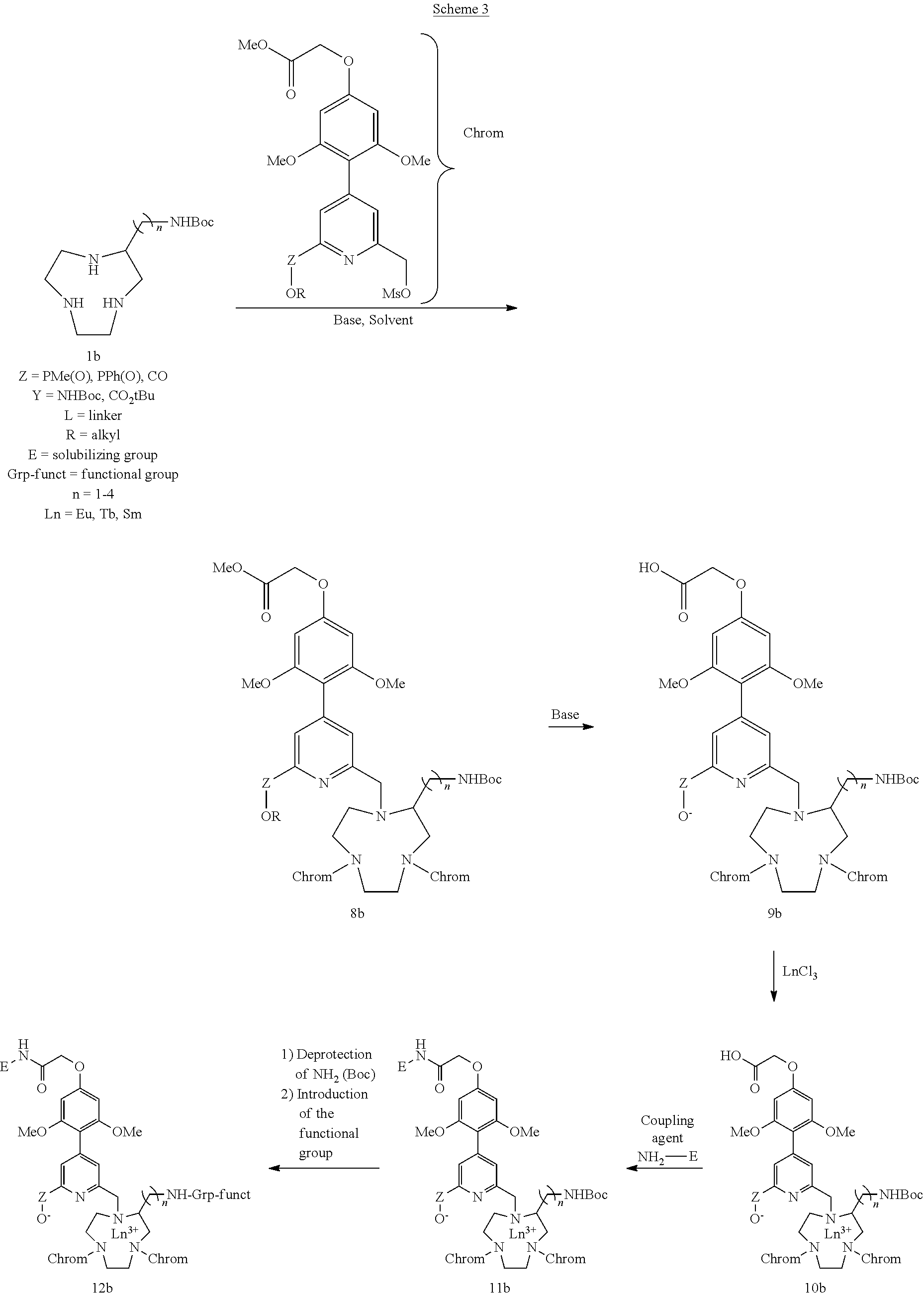

With regard to the tri-antenna complexes, the synthesis is simplified since the amine function that makes it possible to introduce a functional group is fixed directly on the triazacyclononane macrocycle (TACN). The three chromophores are thus introduced in the first step, followed by hydrolysis of the ester functions (carboxylates and phosphinates) and then complexation with the desired lanthanide. The complexes are made soluble by fixing solubilizing groups E on each of the chromophores. After deprotection of the amine, this function is converted to a reactive function allowing bioconjugation.

### 1) Preparation of the Pyridinyl Blocks

The schemes given below (4-12) describe the various synthesis pathways for providing trifunctional pyridinyl derivatives:
in position 2, the complexing function (carboxylic acid or phosphinic acid),
in position 4, a function that makes it possible either to introduce the hydrosolubilizing group (methyl ester function) or else a function that makes it possible to incorporate the functional group (protected amine function and tert-butyl ester function)
and finally in position 6, a methyl alcohol function that is converted to the corresponding mesylate so as to be able to react with the amines of the TACN ring.

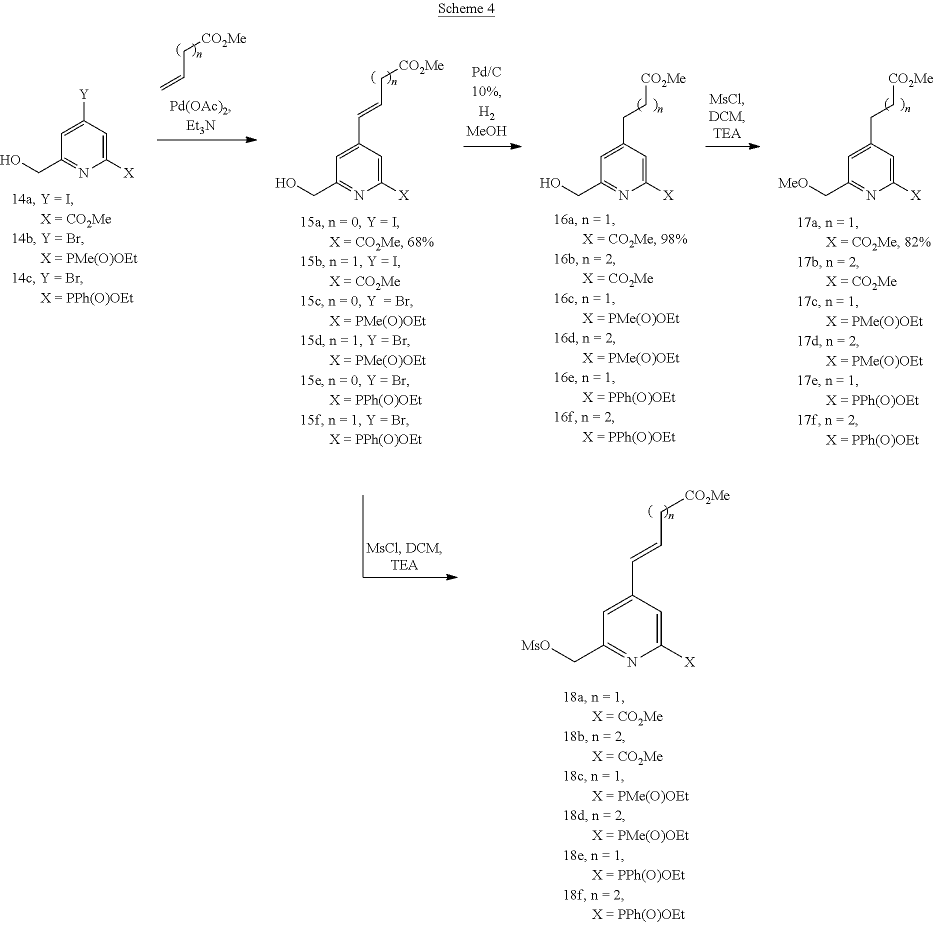

The syntheses of the synthons 14a-c have been described before (cf. applications WO 2013/011236 and WO 2014/111661). Starting from these synthons, the series of compounds 17a-f was obtained by a sequence of three reactions: Heck reaction for creating the carbon-carbon bond between the pyridine derivative and the alkene. This procedure has been described for example in patent application EP-A-2 002 836. Reduction of the double bond by catalytic hydrogenation followed by the mesylation reaction leads to the compounds 17a-f. Alternatively the double bond may be kept to stiffen the system and impose an apical orientation on the hydrosolubilizing groups (18a-f).

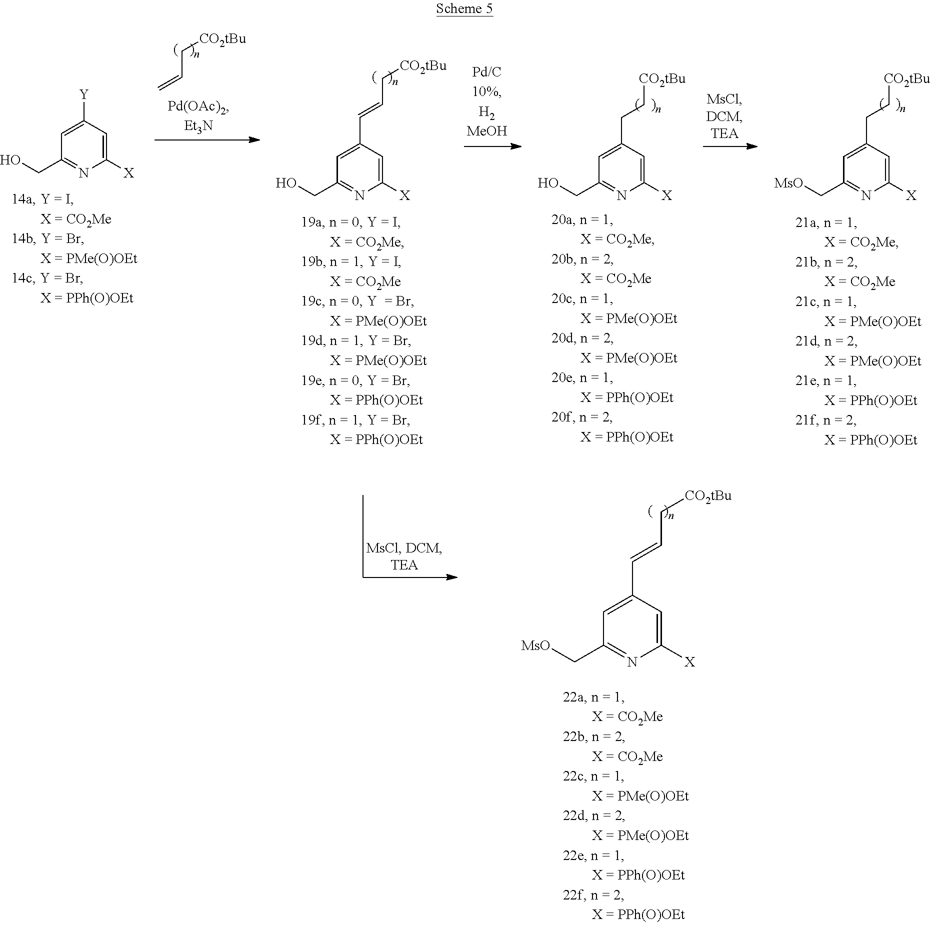

Compounds 21a-f and 22a-f (scheme 5) in the form of tert-butyl ester (analogues of series 17 and 18) were obtained following the same strategy and using the corresponding alkene.

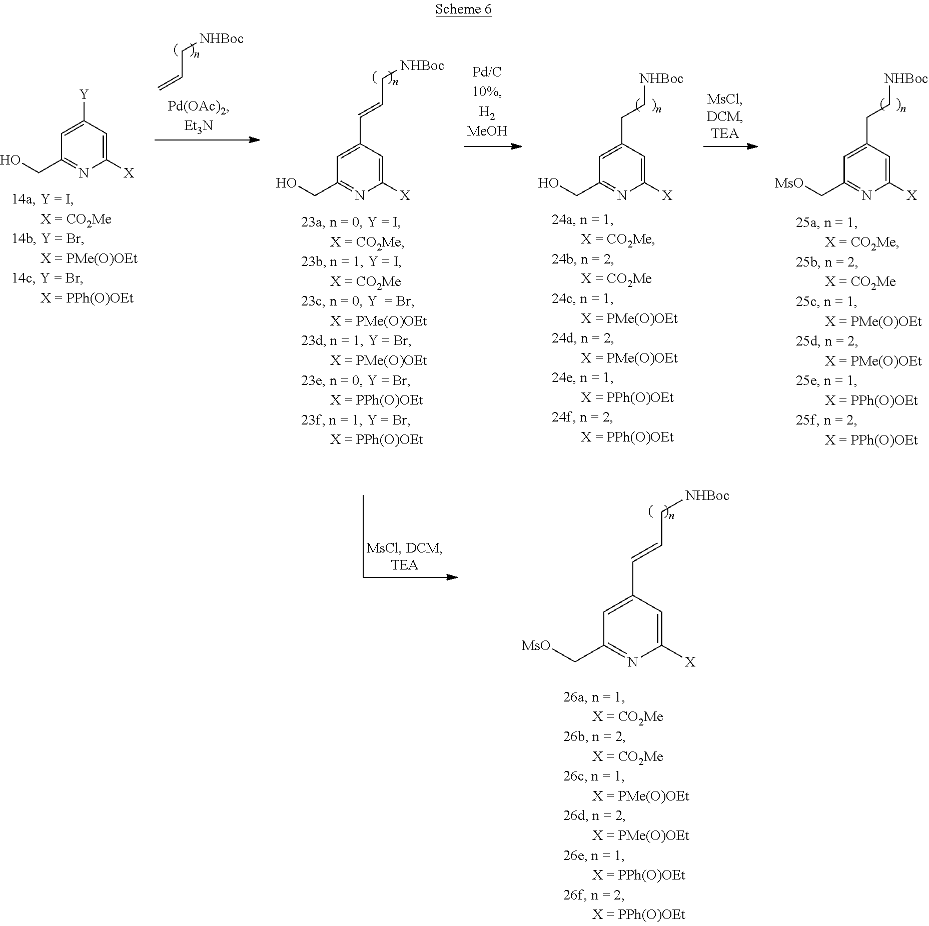

Compounds 25a-f and 26a-f (scheme 6) in NHBoc form (analogues of series 17 and 18) were obtained following the same strategy using the corresponding alkene.

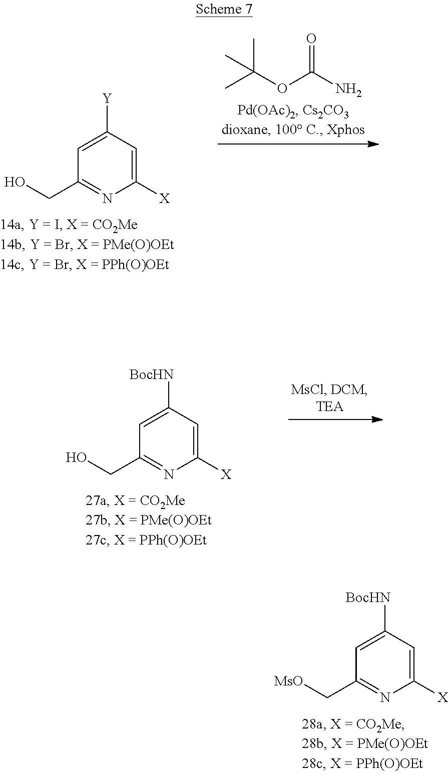

Compounds 28a-c (without carbon chain), analogues of series 25, were prepared according to a similar strategy. Introduction of the NHBoc group was carried out for example using the method described in the review article Tetrahedron Letters 2010, 51, 4445.

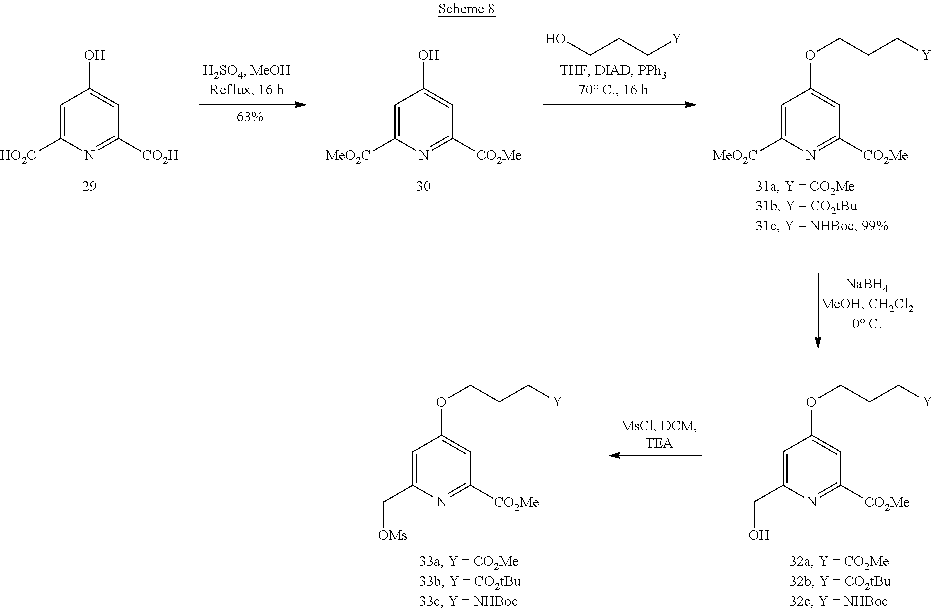

The pyridinyl derivatives, on which an oxygen atom is inserted in position 4 between the aliphatic linker bearing the function (CO₂R or NHBoc) and the aromatic ring (pyridine), were prepared by the method described in scheme 8. Chelidamic acid 29 was esterified in the form of methyl diester and then the linker bearing the function was introduced using a Mitsunobu reaction (procedure described for example in Organic Biomolecular Chemistry 2012, 10, 9183). Mono-reduction using sodium borohydride allows compounds 32a-c to be obtained in the form of monohydric alcohols, which are then converted to corresponding mesylated derivatives 33a-c.

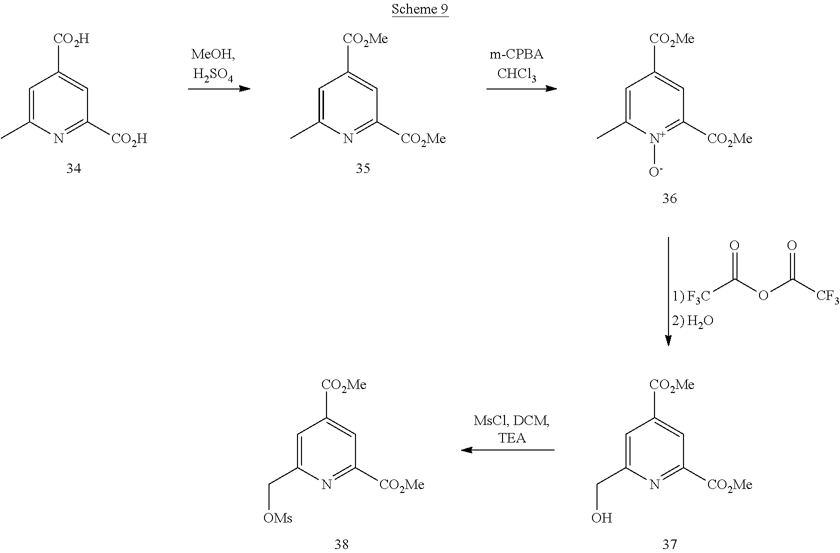

The methyl ester function in position 4 may be fixed directly on the aromatic ring (pyridine). In this case, it is necessary to start from commercial compound 34, which is first esterified. The pyridine is then oxidized in the presence of m-CPBA leading to the corresponding N-oxide derivative 36. The N-oxide function reacts easily with trifluoroacetic anhydride, undergoing a rearrangement, leading after hydrolysis to the methyl alcohol function in position 6. The latter is mesylated in the conventional conditions thus leading to the compound 38.

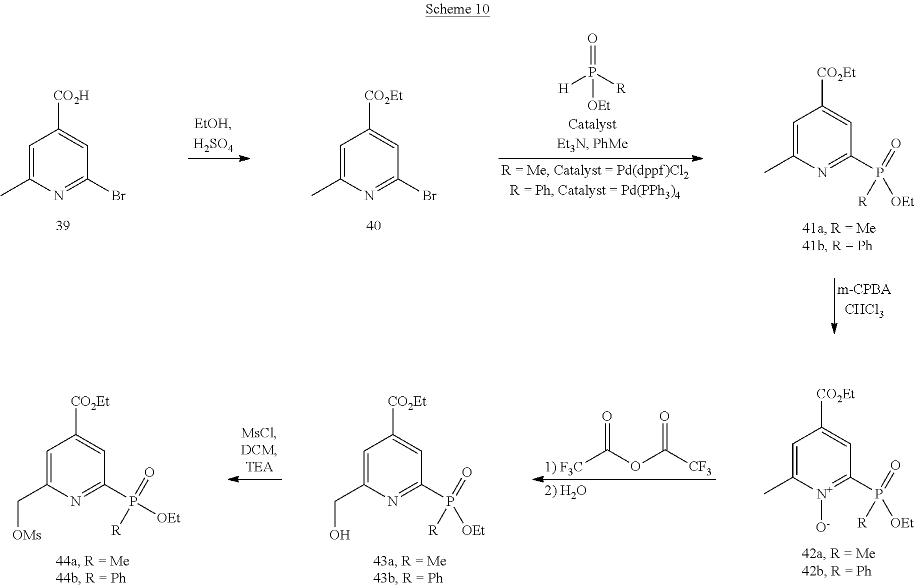

The phosphinate derivatives analogues 44a-b were prepared using compound 39, which is first esterified and then converted to phosphinate ester 41a-b. The rest of the reaction sequence is identical to that used for synthesis of compound 38.

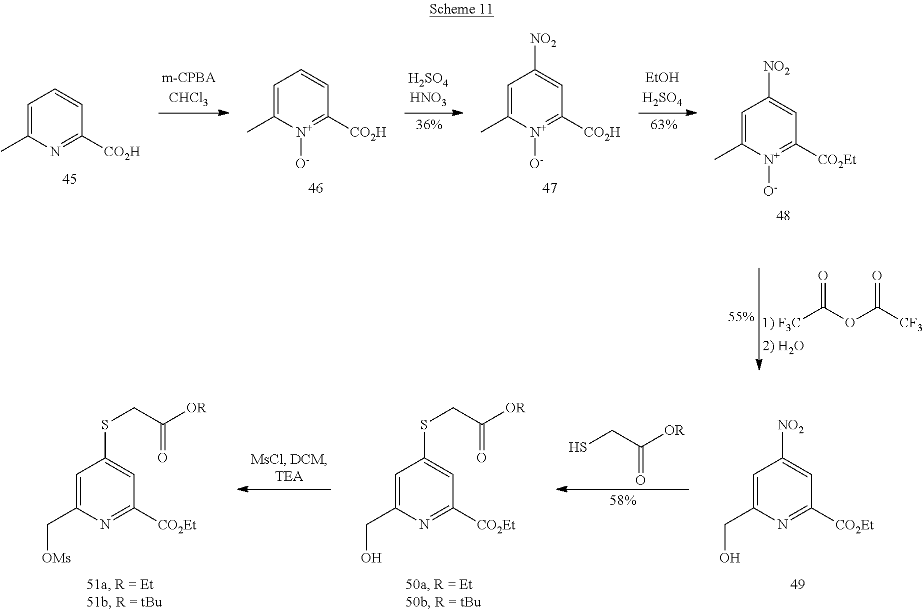

Derivatives 51a-b were prepared according to the reaction sequence described in scheme 11. In this example, the ester functions are introduced using ethyl thioglycolate or tert-butyl thioglycolate.

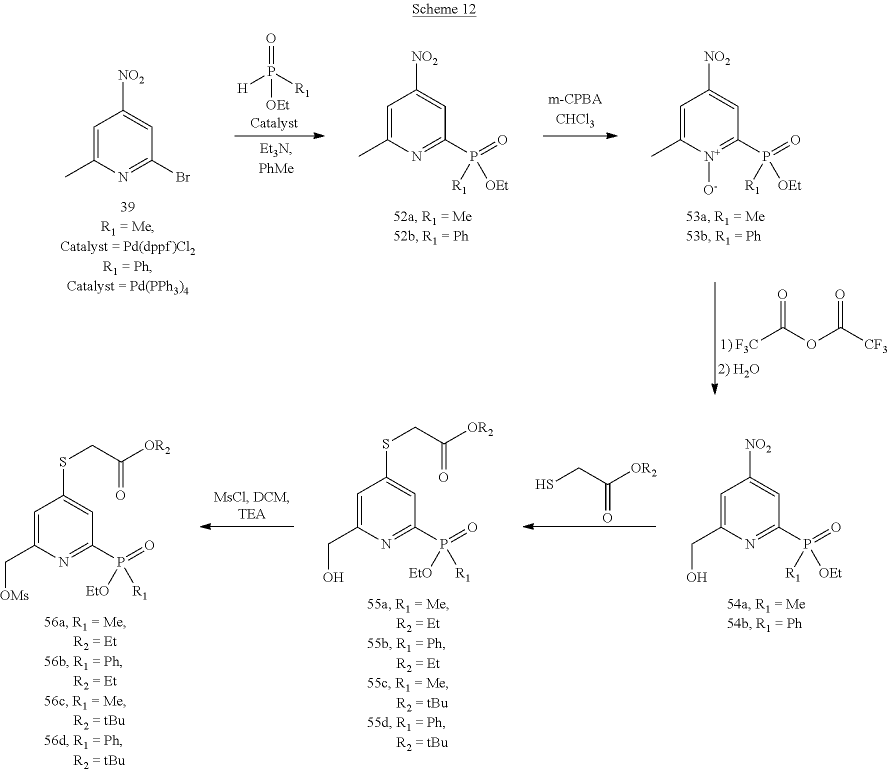

Phosphinate analogues 56a-d are prepared according to the synthesis pathway described in scheme 12.

### 2) Preparation of the Chromophores

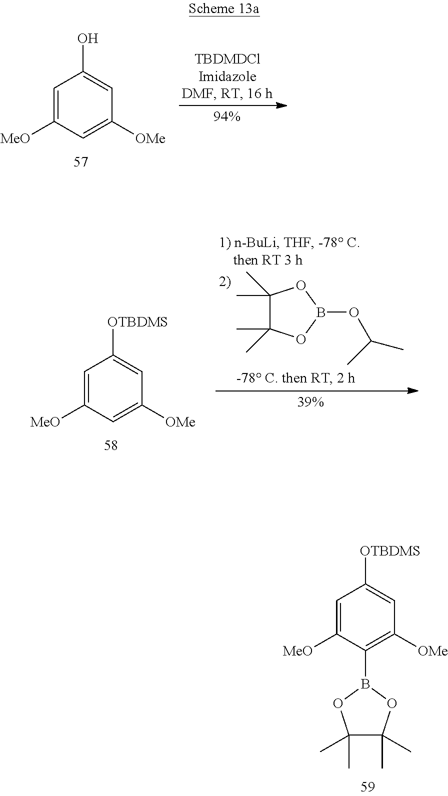 [SMILES: COc1cc(O)cc(OC)c1 ; COc1cc(OC)cc(O[Si](C)(C)C(C)(C)C)c1 ; CC(C)OB1OC(C)(C)C(C)(C)O1 ; COc1cc(O[Si](C)(C)C(C)(C)C)cc(OC)c1B1OC(C)(C)C(C)(C)O1]

The chromophores were synthesized according to schemes 13a-b and 14. The phenol 57 is protected in the form of TBDMS. The next step consists of carrying out selective lithiation in position 4 of the OTBDMS followed by addition of the electrophile (2-isopropoxy-4,4,5,5-tetramethyl-1,3,2-dioxaborolane). Compound 59 was obtained with a yield of 39%.

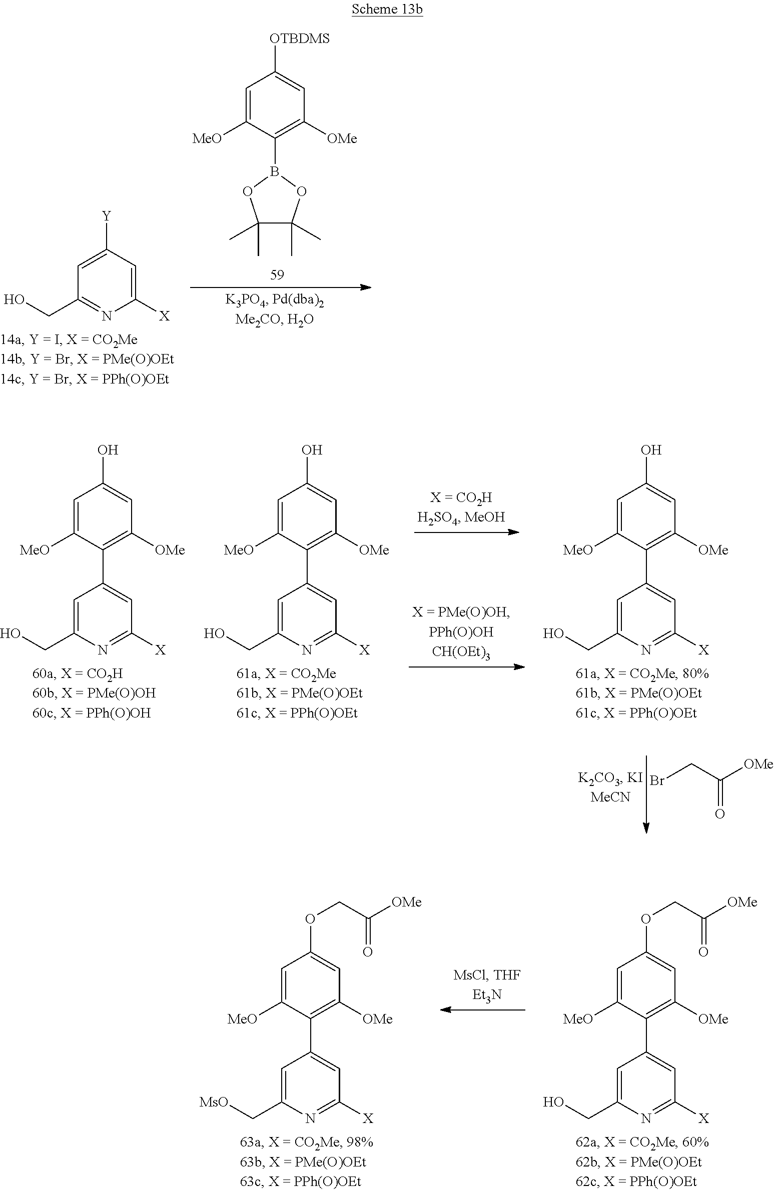

Compound 59 is then coupled via a Suzuki reaction on the pyridine derivatives 14a-c. The conditions of the reaction lead to a mixture of 60a-c (acid form) and 61a-c (ester form). It should be noted that the protective group of the phenol is also removed during this step. This mixture is treated in esterification conditions thus making it possible to convert series 60 into series 61. The phenol function is alkylated (62) and the alcohol function is mesylated, which leads to compounds 63a-c.

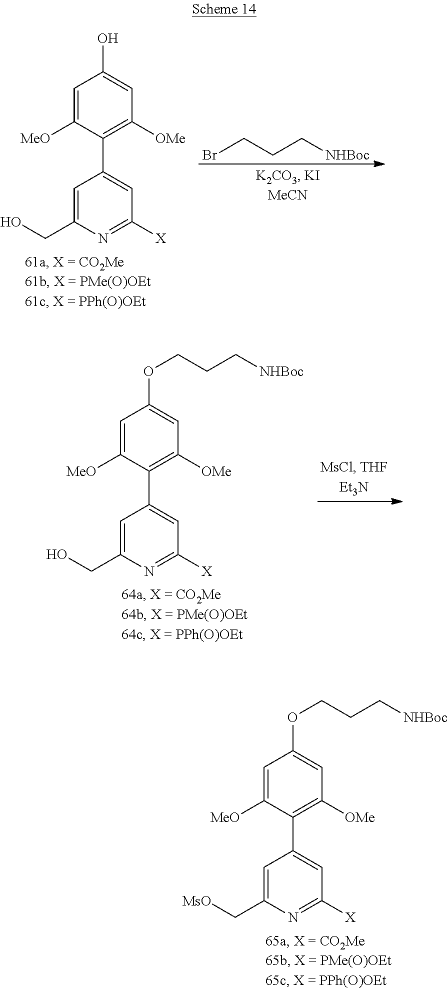

When, for synthesis reasons, the amine function is necessary, it is introduced on the chromophore by alkylating the phenol with bromopropylamine NHBoc, leading to series 64, and then the alcohol function is mesylated (series 65).

### 3) Synthesis of the Mono-Antenna Complexes

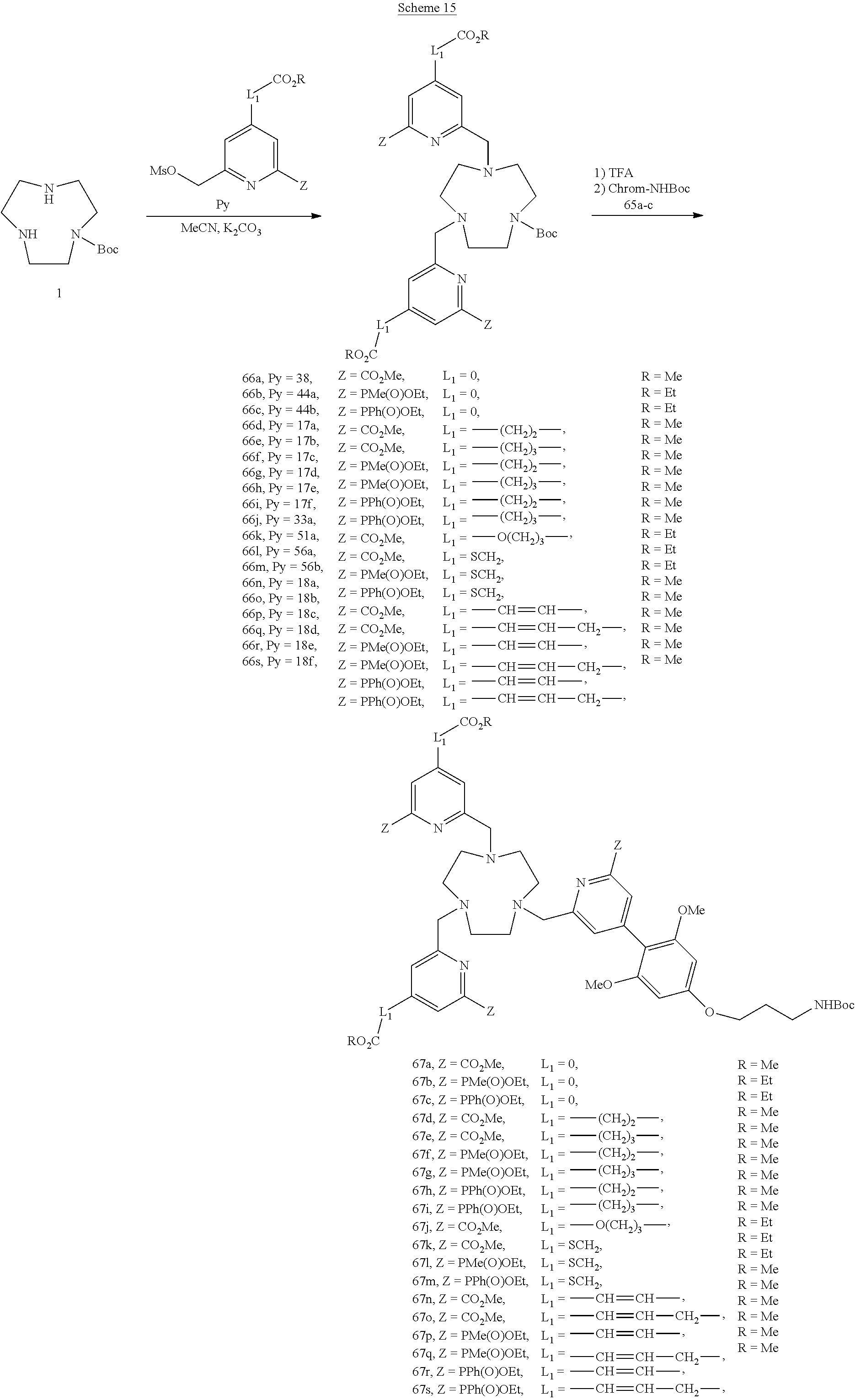

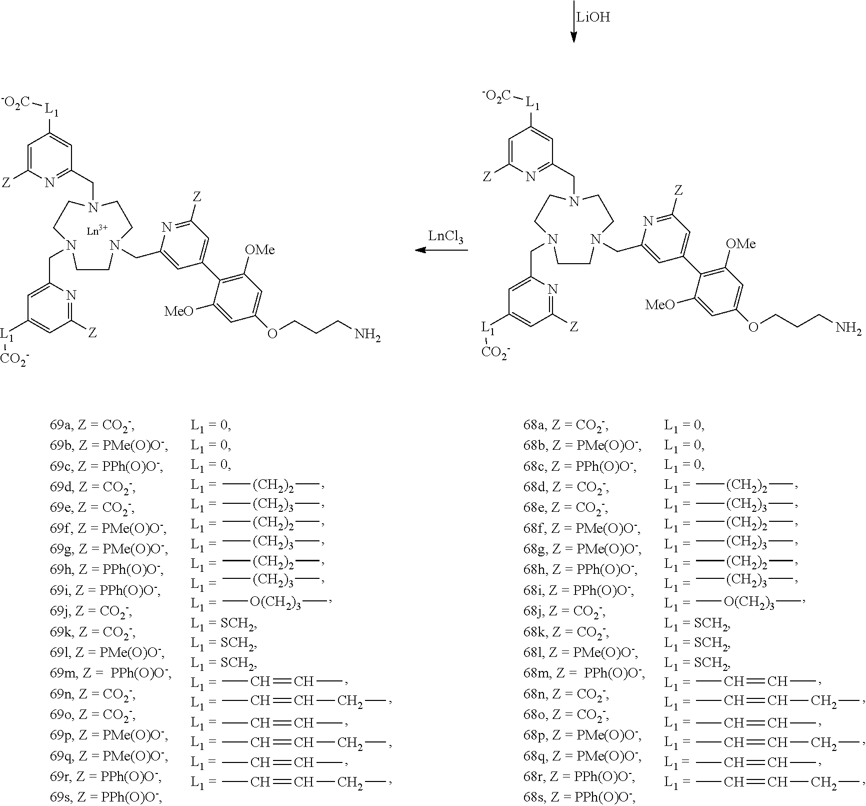

The mono-antenna complexes are synthesized according to scheme 15. Starting from the Boc monoprotected TACN macrocycle, the pyridinyl derivatives (Py) are condensed, leading to compounds 66a-s. The macrocycle is deprotected and the corresponding chromophore (Z identical to those carried by the Py) is introduced on the ligand. The ester functions are hydrolysed (series 68) and the lanthanide (in particular europium or terbium) is complexed in the different ligands, leading to complexes 69a-s.

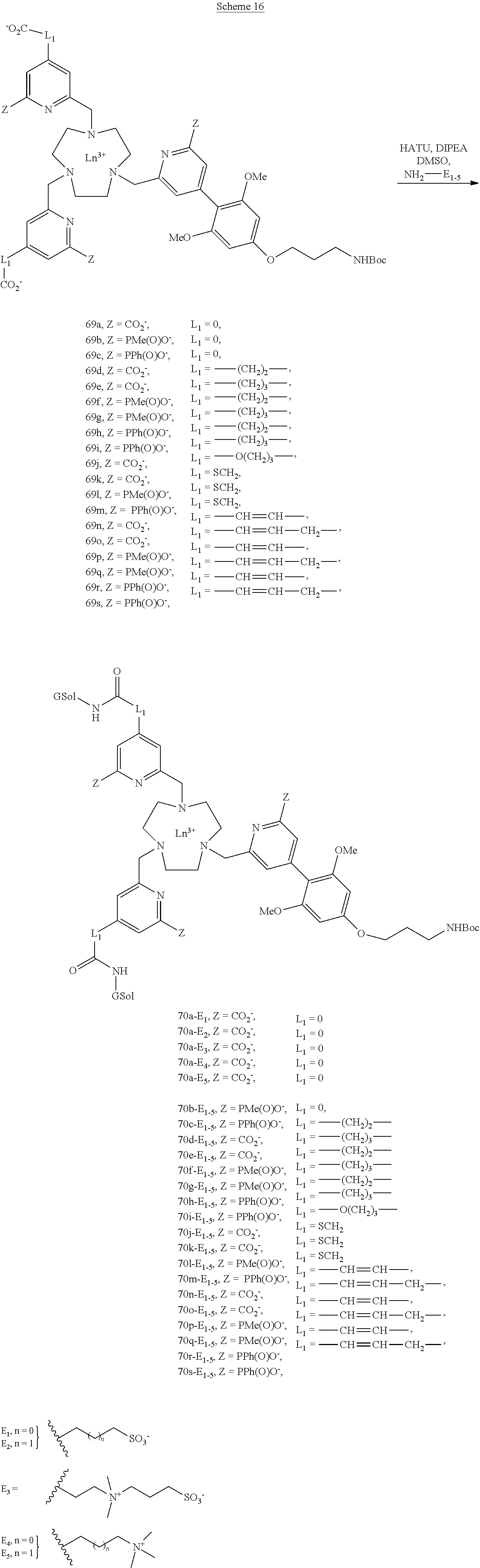

On the series 69a-s, the compounds are made soluble in aqueous media by introducing two hydrosolubilizing groups E₁-E₅: these groups are either of anionic nature (sulfonates, E₁ and E₂) or neutral (zwitterion: sulfobetaines, E₃), or of cationic nature (quaternary ammonium E₄ and E₅).

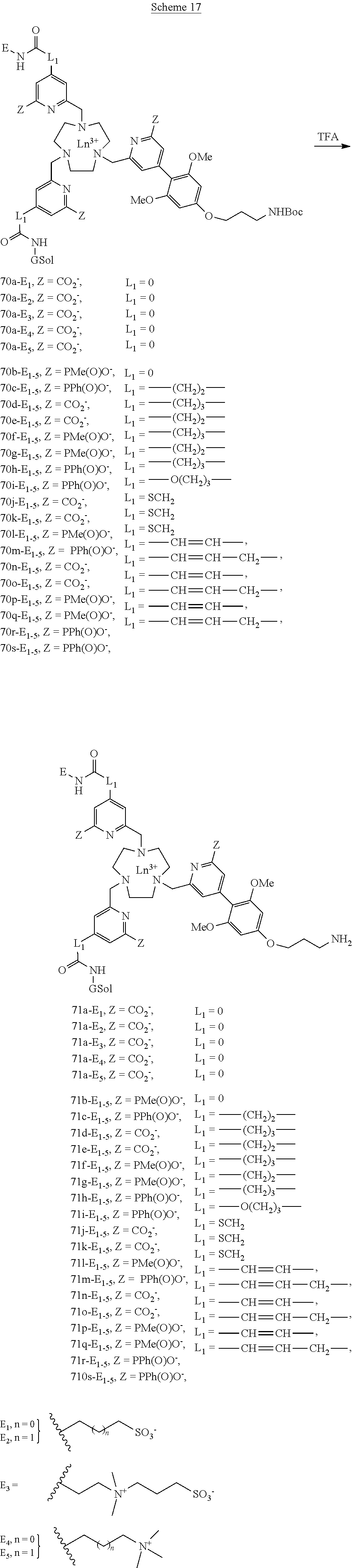

Finally, the Boc group is removed in the presence of trifluoroacetic acid, which leads to the complexes of the invention that are NH₂ functionalized, complexes 71a-E₁₋₅-71s-E₁-5.

### 4) Synthesis of the Di-Antenna Complexes

The synthesis of the di-antenna complexes is described in schemes 18-21.

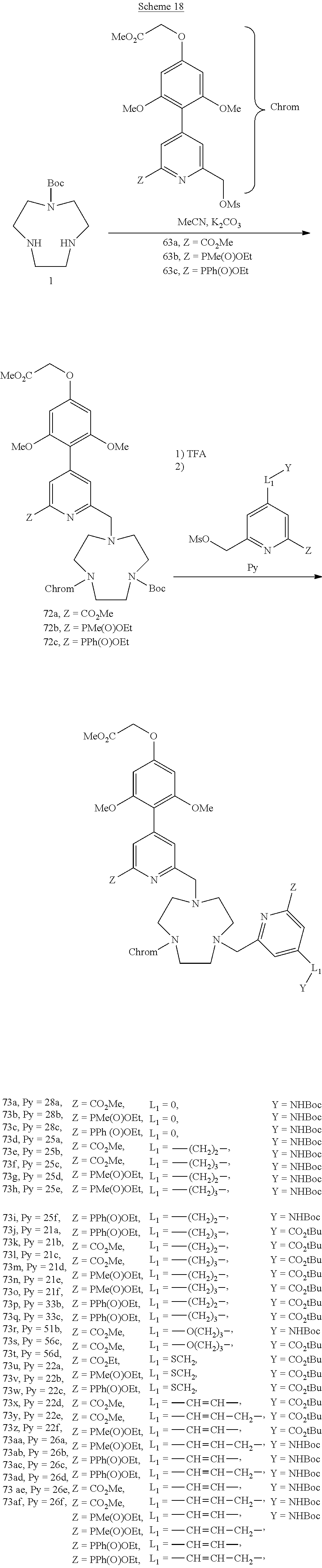

The synthesis begins with the alkylation reaction on the monoprotected TACN with the three types of chromophores: carboxylate, methyl phosphinate and phenyl phosphinate. The Boc protective group is removed and the corresponding pyridines bearing Z identical to the chromophores are introduced on the last alkylation site of the TACN.

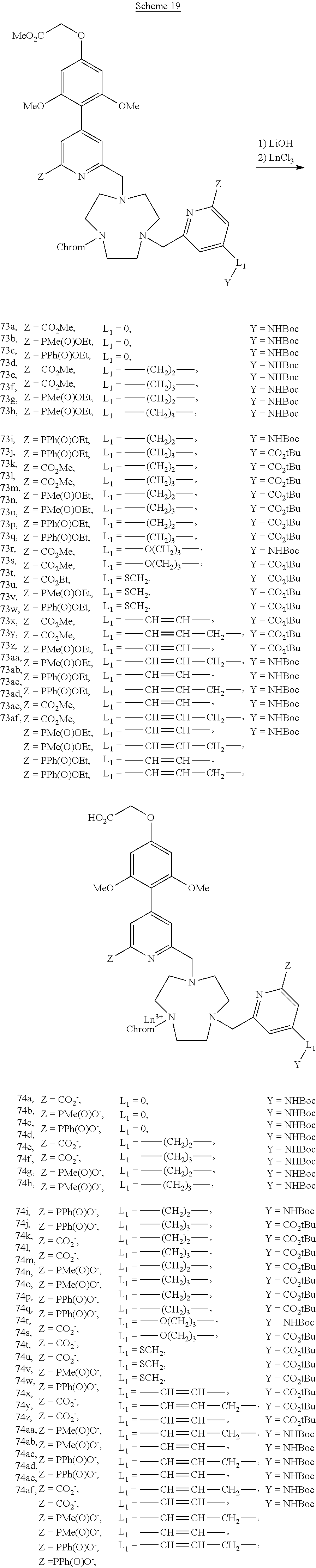

The ligands are hydrolyzed and the lanthanide atom is introduced into the macrocycle, leading to series 74.

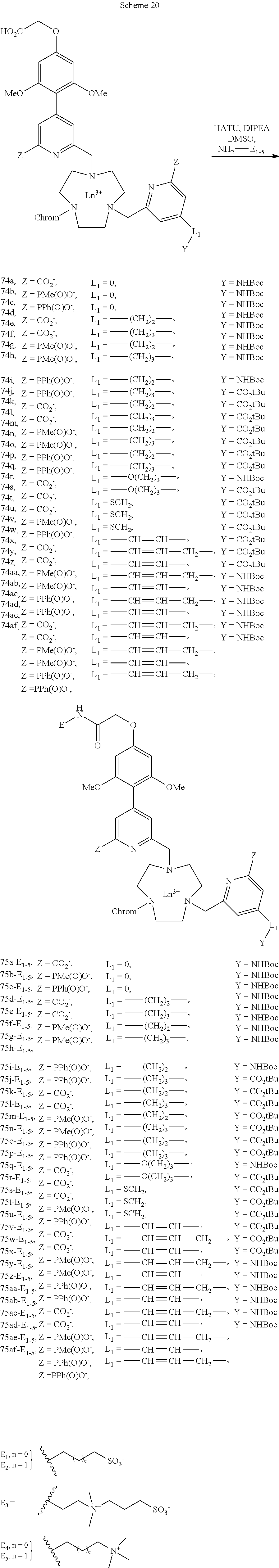

The hydrosolubilizing groups (E₁-E₅) are then introduced on the two chromophores (scheme 20). They are of anionic, neutral or cationic nature.

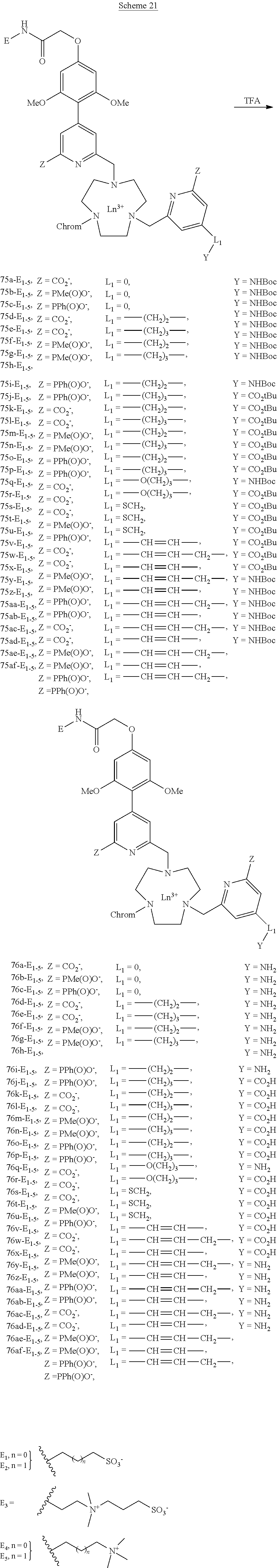

Finally, the Boc or tert-butyl ester group is then removed in the presence of trifluoroacetic acid, leading to compounds 76a-af (scheme 21).

### 5) Synthesis of the Tri-Antenna Complexes

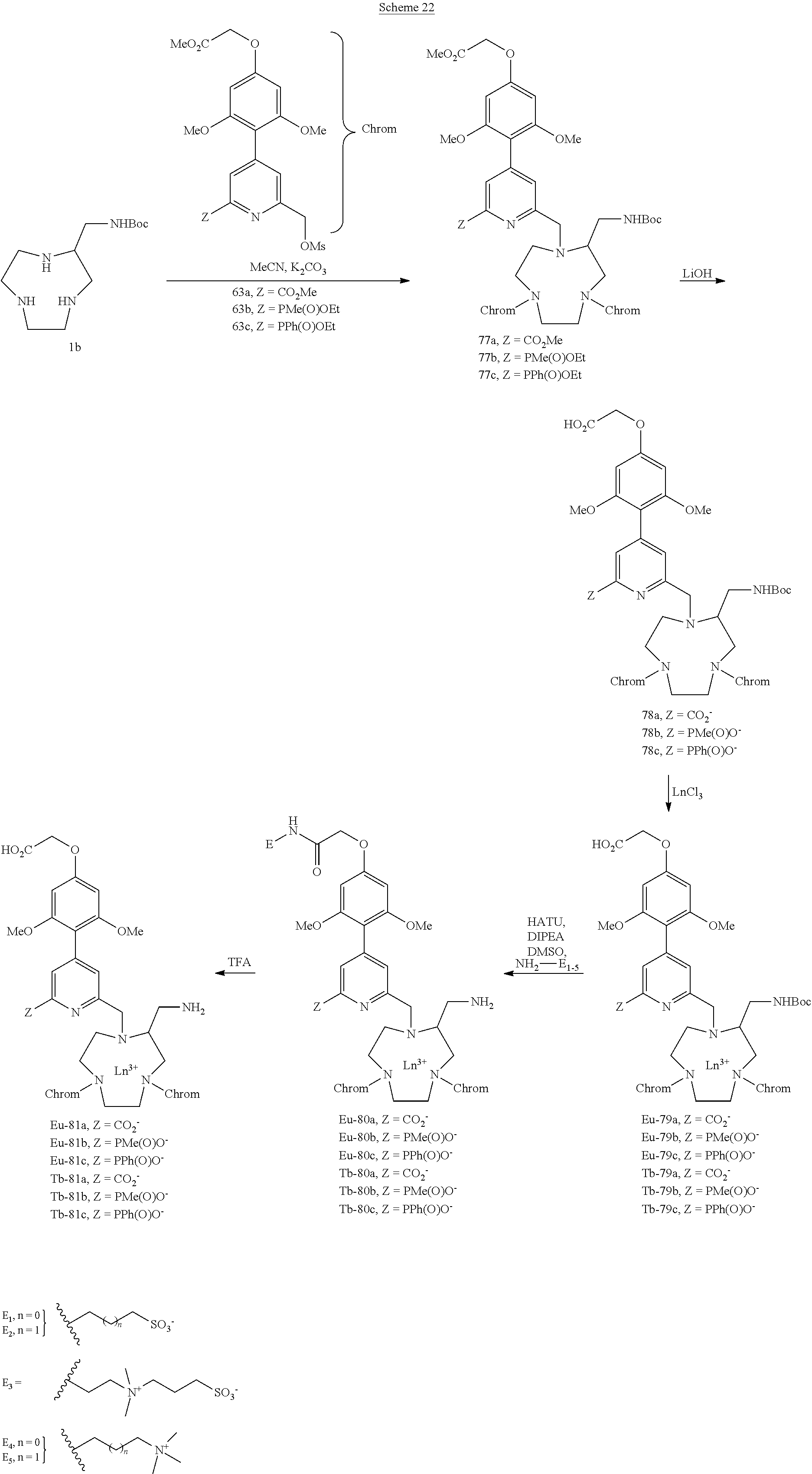

The tri-antenna complexes were synthesized according to the reaction scheme described in scheme 22. The various mesylated pyridines (63a-c) are condensed on the monosubstituted TACN 1b. The ligands 77a-c obtained are hydrolyzed in the presence of lithium hydroxide and then brought into contact with the corresponding lanthanide salts, which leads either to the europium complexes Eu-79a-c or to the terbium complexes Tb-79a-c. After introduction of the hydrosolubilizing groups E₁-E₅, the Boc group is removed in the presence of trifluoroacetic acid, which leads to the complexes Eu-81a-c and Tb-81a-c.

To demonstrate the efficacy of the complexes of the invention Eu-81a-E₂, Tb-81a-E₂, Tb-81a-E₄, they were compared with complexes of the prior art 82a and 82b comprising trimethoxyphenylpyridine antennas. The results of the tests are presented in the experimental section.

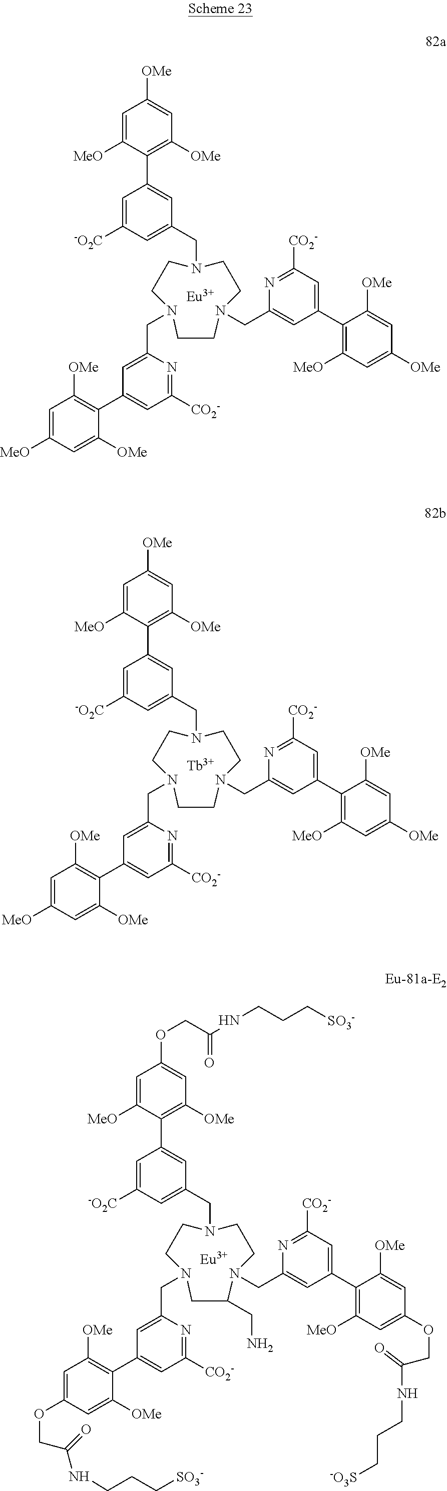

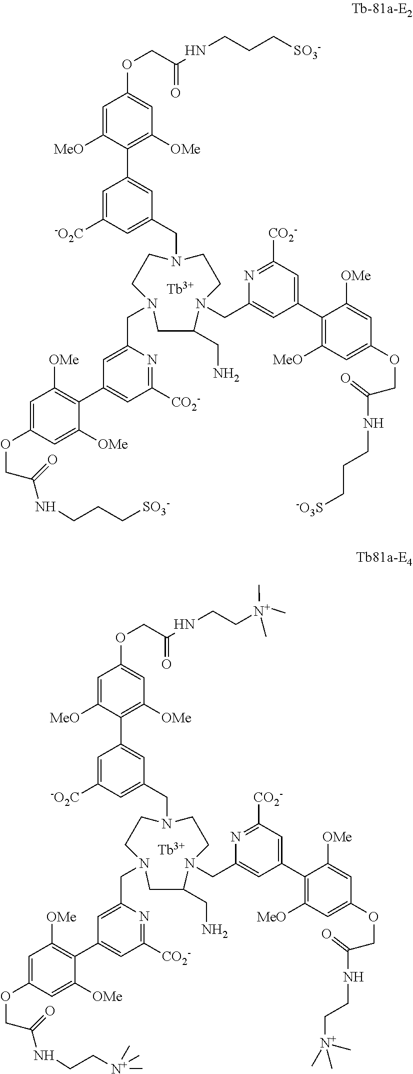

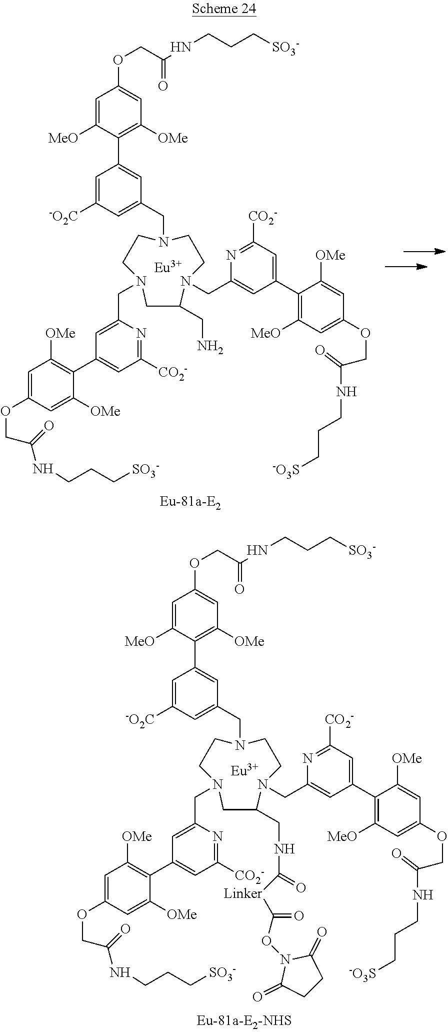

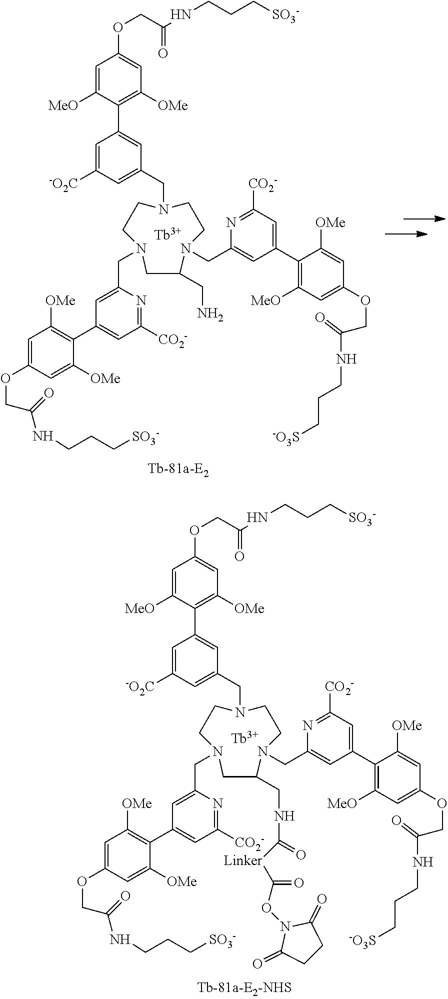

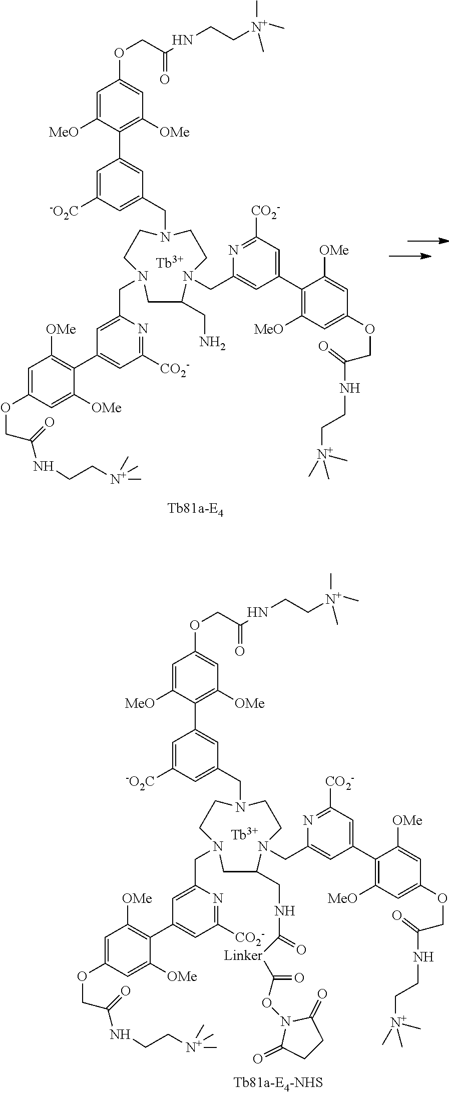

Three of the complexes of the invention were converted into corresponding NHS functionalized complexes (scheme 24). These three complexes are usable for labelling a protein for example and more particularly an antibody.

### Experimental Section

### Abbreviations Used:

EtOAc: ethyl acetate
AcOH: acetic acid
Boc: tert-butyloxycarbonyl
n-BuLi: n-butyllithium
CDCl₃: deuterated chloroform
CHCl₃: chloroform
CH(OEt)₃: ethyl orthoformate
Cs₂CO₃: cesium carbonate
CuI: copper(I) iodide
DCM/CH₂Cl₂: dichloromethane
DIAD: diisopropyl azodicarboxylate
DMF: dimethylformamide
DIPEA: diisopropylethylamine
DMSO: dimethylsulfoxide
Et: ethyl
ESI+: positive-mode electrospray ionization
EtOH: ethanol
h: hour
HATU: (O-(7-azabenzotriazol-1-yl)-N,N,N′,N′-tetramethyluronium hexafluorophosphate)
HNO₃: nitric acid
HPLC: high-performance liquid chromatography
H₂O: water
H₂O₂: hydrogen peroxide
H₂SO₄: sulfuric acid
d: day
K₂CO₃: potassium carbonate
KI: potassium iodide
K₃PO₄: potassium phosphate
LC-MS: high-performance liquid chromatography coupled to mass spectrometry
LiOH: lithium hydroxide
LnCl₃: lanthanide chloride
m-CPBA: metachloroperbenzoic acid
Me: methyl
MeCN: acetonitrile
Me₂CO: acetone
MeOH: methanol
min: minute
Ms: mesyl
MsCl: mesyl chloride/methanesulfonyl chloride
NaBH₄: sodium borohydride
NaH: sodium hydride
Pd/C: palladium on charcoal
Pd(dba)₂: bis(dibenzylideneacetone)palladium(0)
Pd(dppf)Cl₂: bis(diphenylphosphino)ferrocene]dichloropalladium(II)
Pd(OAc)₂: palladium(II) acetate
Pd(PPh₃)₄: tetrakis(triphenylphosphine)palladium(0)
Ph: phenyl
PhMe: toluene
PPh₃: triphenylphosphine
mp: melting point
Py: pyridine
Rf: solvent front
Rt: retention time
RT: room temperature
TEA/Et₃N: triethylamine
TFA: trifluoroacetic acid
THF: tetrahydrofuran
TBDMSCl: tert-butyldimethylsilyl chloride
Ts: tosyl
TSTU: O—(N-succinimidyl)-1,1,3,3-tetramethyluronium tetrafluoroborate
UPLC-MS: ultrahigh-performance liquid chromatography coupled to mass spectrometry
Xphos: 2-dicyclohexylphosphino-2′,4′,6′-triisopropylbiphenyl

### Chromatography

Analytical and preparative high-performance liquid chromatography (HPLC) was carried out with two instruments:
Analytical HPLC: ThermoScientific, P4000 quaternary pump, UV 1000 detector with deuterium lamp (190-350 nm), Waters XBridge C18 analytical column, 3.5 μm, 4.6×100 mm.
Preparative HPLC: Shimadzu, 2 LC-8A pumps, Varian ProStar diode-array UV detector, Waters XBridge prep. C18 preparative column, 5 μm: 19×100 mm or 50×150 mm.

Analytical ultrahigh-performance liquid chromatography (UPLC) was carried out on a Waters Acquity HClass instrument with, as detector, either a diode-array UV detector of the PDA type or a quadrupolar simple mass detector of the SQD2 type. The probe used is positive-mode electrospray: capillary voltage at 3.2 kV—cone voltage at 30 V.

Silica column chromatography was carried out on Merck 60 silica gel (0.040-0.063 mm). Alumina column chromatography was carried out on Sigma-Aldrich aluminum oxide, neutral, activated, Brochmann I.

### Spectroscopy

Nuclear Magnetic Resonance (NMR)

The NMR spectra (1H, ¹³C and ³¹P) were recorded using a Bruker Advance 400 MHz NanoBay spectrometer (9.4 tesla magnet), equipped with a BBFO measurement probe, multicore with diameter of 5 mm, Z gradient and ²H lock. The chemical shifts (5) are expressed in parts per million (ppm). The following abbreviations are used:

s: singlet, bs: broad singlet, app s: apparent singlet, d: doublet, t: triplet, q: quadruplet, m: multiplet, dd: doublet of doublets, dt: doublet of triplets, dq: doublet of quadruplets, ddd: doublet of doublet of doublets, AB: AB system.

Mass Spectrometry (LRMS)

The mass spectra (LC-MS) were recorded using a Waters ZQ 2000 single quadrupole multimode-source ESI/APCI spectrometer equipped with a Waters XBridge C18 column, 3.5 μm, 4.6×100 mm or else a single quadrupole mass spectrum of the SQD2 type.

High-Resolution Mass Spectrometry (HRMS)

The analyses were carried out with a QStar Elite mass spectrometer (Applied Biosystems SCIEX) equipped with a pneumatically assisted atmospheric pressure ionization (API) source. The sample was ionized in positive electrospray mode in the following conditions: electrospray voltage (ISV): 5500 V; orifice voltage (OR): 20 V; nebulizing gas pressure (air): 20 psi. The high-resolution mass spectrum (HRMS) was obtained with a time-of-flight (TOF) analyzer. Exact measurement of mass was carried out in triplicate with double internal calibration.

### Gradient A

Waters Acquity C18 column, 300 Å, 1.7 μm, 2.1×50 mm−A/water 0.1% formic acid B/acetonitrile 0.1% formic acid t=0 min 5% B−t=0.2 min 5% B−t=5 min 100% B−0.6 mL·min⁻¹.

### Gradient B

Waters XBridge C18 column, 5 μm, 50×150 mm−A/water 25 mM TEAAc pH 7 B/acetonitrile t=0 min 10% B−t=19 min 60% B−100 mL·min⁻¹.

### Gradient C

Waters Acquity C18 column, 300 Å, 1.7 μm, 2.1×50 mm−A/water 5 mM ammonium acetate B/acetonitrile t=0 min 5% B−t=0.2 min 5% B−t=5 min 100% B—0.6 mL·min⁻¹.

### Gradient D

Waters XBridge C18 column, 5 μm, 20×100 mm−A/water 25 mM TEAAc pH 7 B/acetonitrile t=0 min 5% B−t=19 min 60% B−20 mL·min⁻¹.

### Gradient E

Waters XBridge C18 column, 5 μm, 20×100 mm−A/water 25 mM TEAAc pH 7 B/acetonitrile t=0 min 2% B−t=19 min 40% B−20 mL·min⁻¹.

### Gradient F

Waters XBridge C18 column, 5 μm, 20×100 mm−A/water 25 mM TEAAc pH 6 B/acetonitrile t=0 min 2% B−t=19 min 40% B−20 mL·min⁻¹.

### Gradient G

Waters XBridge C18 column, 5 μm, 50×150 mm−A/water 0.2% TFA B/acetonitrile t=0 min 10% B−t=12 min 50% B−80 mL·min⁻¹.

### Gradient H

Waters XBridge C18 column, 5 μm, 50×150 mm−A/water 0.2% TFA B/acetonitrile t=0 min 30% B−t=20 min 100% B−80 mL·min⁻¹.

### EXAMPLES

Compound 1: compound 1 was prepared according to the procedure described in applications WO 2013/011236 and WO 2014/111661.

Compounds 14a-14c: compounds 14a-14c were prepared according to the procedure described in applications WO 2013/011236 and WO 2014/111661.

Compound 15a: in a 100-mL Schlenk flask, compound 14a (440 mg, 1.5 mmol) was dissolved in anhydrous DMF (10 mL) to give a colorless solution. Tri(o-tolyl)phosphine (91 mg, 0.3 mmol), Pd(OAc)₂ (33.7 mg, 0.15 mmol), TEA (0.314 mL, 2.252 mmol) and then methyl acrylate (0.203 mL, 2.252 mmol) were added to the reaction mixture in one go. The reaction was stirred at 70° C. for 5 h. The progress of the reaction was monitored by UPLC-MS (gradient A). After this time, the reaction was complete. The reaction mixture was concentrated under reduced pressure, diluted in EtOAc (50 mL), washed with water (2×50 mL) and then with water saturated with NaCl (50 mL). The organic phase was dried over MgSO₄, filtered and concentrated under reduced pressure. The crude product was purified by silica column chromatography using a DCM/MeOH solvent gradient from 100/0 to 99/1 to give compound 15a (233 mg, 62%) in the form of white powder.

mp=156.4-156.9° C.—HPLC gradient A—Rt=2.03 min—[M+H]⁺, m/z 251.9—Rf=0.41 (silica, dichloromethane—methanol 96:4—HRMS (ESI+) calculated for C₁₂H₁₄NO₅⁺ [M+H]*, m/z 252.0866, found: 252.0868—¹H NMR (400 MHz, CDCl₃) δ: 8.13 (s, 1H, Py H⁵), 7.67 (d, J=16.2 Hz, 1H), 7.65 (s, 1H, Py H³), 7.19 (dd, J=; 16.2 Hz, 2H, HC═CH), 6.71 (d, J=16.2 Hz, 1H), 4.91 (s, 2H, CH₂—OH), 4.04 (s, 3H, Py-COOMe), 3.85 (s, 3H, COOMe), 3.49 (bs, 1H, OH); ¹³C NMR (100 MHz, CDCl₃) δ: 166.20 (COOMe), 165.24 (Py-COOMe), 161.59 (Py C²), 147.97 (Py-C═C), 143.74 (Py C⁶), 140.92 (Py C⁴), 123.77 (Py C³), 122.08 (Py C⁵), 121.86 (Py-C═C), 64.68 (CH₂—OH), 53.10 (Py-CO₂CH₃), 52.19 (CO₂CH₃).

Compounds 15b-15f: these compounds were prepared according to the same procedure as that used for the synthesis of 15a using the corresponding alkenes.

Compound 16a: in a 50-mL flask, compound 15a (233 mg, 0.927 mmol) was dissolved in MeOH (10 mL) to give a colorless solution. Pd/C 10% (23.69 mg, 0.022 mmol) was added to the reaction mixture in one go. The reaction was stirred at RT with bubbling of dihydrogen for 2 h. The progress of the reaction was monitored by UPLC-MS (gradient A). After this time, the reaction was complete. The reaction mixture was filtered on a 22 μm nylon filter, and evaporated to dryness to give compound 16a (231 mg, 98%) in the form of white powder. mp=133.2-136.4° C.—HPLC gradient A—Rt=1.86 min—[M+H]⁺, m/z 253.2—HRMS (ESI+) calculated for C₁₂H₁₆NO₅⁺ [M+H]⁺, m/z 254.1023, found: 254.1024—¹H NMR (400 MHz, CDCl₃) δ: 7.9 (s, 1H, Py H⁵), 7.41 (s, 1H, Py H³), 4.85 (s, 2H, CH₂—OH), 4.01 (s, 3H, Py-COOMe), 3.69 (s, 3H, COOMe), 3.05 (t, J=7.6 Hz, 2H, Py-CH₂—CH₂), 2.71 (t, J=7.6 Hz, 2H, Py-CH₂-CH₂); ¹³C NMR (100 MHz, CDCl₃) δ: 172.41 (COOMe), 165.65 (Py-COOMe), 160.49 (Py C²), 151.67 (Py C⁴), 147.22 (Py C⁶), 140.92 (Py C³), 123.96 (Py C³), 123.9 (Py C⁵), 64.62 (CH₂—OH), 52.91 (Py-CO₂CH₃), 51.91 (CO₂CH₃), 33.97 (Py-CH₂-CH₂), 30.07 (Py-CH₂—CH₂).

Compounds 16b-16f: these compounds were prepared according to the same procedure as that used for the synthesis of 16a.

Compound 17a: in a 100-mL flask, compound 16a (231 mg, 0.912 mmol) was dissolved in anhydrous THF (30 mL) to give a colorless solution. TEA (0.127 mL, 0.912 mmol) and then MsCl (72 μL, 0.912 mmol) were added in one go to the reaction mixture placed in an ice bath. The mixture was warmed to RT and stirred for 15 min. The progress of the reaction was monitored by UPLC-MS (gradient A). After this time, the reaction was complete. The reaction mixture was concentrated under reduced pressure, diluted in DCM (50 mL), washed with water (2×25 mL) and then with water saturated with NaCl (20 mL). The organic phase was dried over MgSO₄, filtered and concentrated under reduced pressure to give compound 17a (249 mg, 82%) in the form of white powder. HPLC gradient A—Rt=3.2 min—[M+H]⁺, m/z 332.3—Rf=0.23 (silica, dichloromethane—methanol 98:2—HRMS (ESI+) calculated for C₁₃H₁₈NO₇S⁺ [M+H]⁺, m/z 332.0799, found: 332.0799—¹H NMR (400 MHz, CDCl₃) δ: 7.98 (s, 1H, Py H⁵), 7.54 (s, 1H, Py H³), 5.41 (s, 2H, CH₂-OMs), 4.00 (s, 3H, Py-COOMe), 3.69 (s, 3H, COOMe), 3.16 (s, 3H, OMs), 3.07 (t, J=7.5 Hz, 2H, Py-CH₂—CH₂), 2.72 (t, J=7.5 Hz, 2H, Py-CH₂-CH₂); ¹³C NMR (100 MHz, CDCl₃) δ: 172.23 (COOMe), 165.27 (Py-COOMe), 154.56 (Py C²), 152.46 (Py C⁴), 147.93 (Py C⁶), 125.19 (Py C³), 125.03 (Py C⁵), 70.97 (CH₂-OMs), 53.08 (Py-CO₂CH₃), 51.94 (CO₂CH₃), 38.05 (CH₂—OSO₂CH₃), 33.87 (Py-CH₂-CH₂), 30.07 (Py- CH₂—CH₂).

Compounds 17b-17f: these compounds were prepared according to the same procedure as that used for the synthesis of 16a.

Compounds 18a-18f: these compounds were prepared according to the same procedure as that used for the synthesis of 17a.

Compounds 19a-19f: these compounds were prepared according to the same procedure as that used for the synthesis of 15a.

Compounds 20a-20f: these compounds were prepared according to the same procedure as that used for the synthesis of 16a.

Compounds 21a-21f: these compounds were prepared according to the same procedure as that used for the synthesis of 17a.

Compounds 22a-22f: these compounds were prepared according to the same procedure as that used for the synthesis of 17a.

Compounds 23a-23f: these compounds were prepared according to the same procedure as that used for the synthesis of 15a using the corresponding alkenes.

Compounds 24a-24f: these compounds were prepared according to the same procedure as that used for the synthesis of 16a.

Compounds 25a-25f: these compounds were prepared according to the same procedure as that used for the synthesis of 17a.

Compounds 26a-26f: these compounds were prepared according to the same procedure as that used for the synthesis of 17a.

Compounds 27a-c: compounds 27a-c were prepared according to the procedure described in the article: Tetrahedron Letters 2010, 51, 4445.

Compounds 28a-28c: these compounds were prepared according to the same procedure as that used for the synthesis of 17a.

Compound 29: this compound is available commercially.

Compound 30: compound 30 was prepared according to the procedure described in the article: Dalton Transactions 2010, 39, 707.

Compounds 31a-31c: compounds 31a-31c were prepared according to the procedure described in the article: Organic Biomolecular Chemistry 2012, 10, 9183.

Compounds 32a-32c: compounds 32a-32c were prepared according to the procedure described in the article: Journal of Organic Chemistry 2010, 75, 7175.

Compounds 33a-33c: compounds 33a-33c were prepared according to the procedure described in the article: Journal of Organic Chemistry 2010, 75, 7175.

Compound 34: this compound is available commercially.

Compound 35: compound 35 was prepared according to the procedure described in the article: Bioorganic Chemistry 2014, 57, 148.

Compound 36: compound 36 was prepared according to the procedure described in the article: Carbohydrate Research 2013, 372, 35.

Compound 37: compound 37 was prepared according to the procedure described in application WO 2014/111661.

Compound 38: the compound was prepared according to the same procedure as that used for the synthesis of 17a.

Compound 39: available commercially.

Compound 40: compound 40 was prepared according to the procedure described in the article: Bioorganic Chemistry 2014, 57, 148.

Compound 41a-b: compounds 41a-b were prepared according to the procedure described in application WO 2014/111661 using the corresponding catalyst.

Compound 42a-b: compounds 42a-b were prepared according to the same procedure as that used for the synthesis of 36.

Compound 43a-b: compounds 43a-b were prepared according to the same procedure as that used for the synthesis of 37.

Compound 44a-b: compounds 44a-b were prepared according to the same procedure as that used for the synthesis of 17a.

Compound 45: this compound is available commercially.

Compound 46: compound 46 was prepared according to the procedure described in the article: Chemistry—A European Journal, 2014, 20, 3610.

Compound 47: compound 46 (0.313 g, 2.04 mmol) was dissolved in H₂SO₄ (11 mL) at RT and then the solution was cooled in an ice bath. HNO₃ (9.7 mL) was added dropwise to this mixture and the solution was heated at 100° C. for 2 d. The mixture was cooled to RT and then poured into crushed ice (100 g). After 1 h, the aqueous phase was extracted with CH₂Cl₂ (3×50 mL), the organic phases were combined, dried over MgSO₄ and the crude product was purified by silica column chromatography using a solvent mixture (CH₂Cl₂—AcOH, 98/2) to give a white solid (224 mg, 56%). R_{f} (CH₂Cl₂/AcOH, 98/2)=0.38; mp: 147° C.; ¹H NMR (400 MHz, CDCl₃, δ): 16.49 (s, 1H, COOH), 9.08 (s, 1H, H³), 8.36 (s, 1H, H⁵), 2.75 (s, 3H, py-CH₃); ¹³C NMR (101 MHz, CDCl₃, δ): 159.4 (COOH), 152.4 (C⁶), 144.4 (C⁴), 138.7 (C²), 123.1 (C⁵), 121.7 (C³), 18.4 (py-CH₃); MS Calculated for C₇H₇N₂O₅ 199.036. Found 199.035 [M+H]⁺.

Compound 48: compound 47 (2.9, 14.7 mmol) was dissolved in anhydrous MeOH (3 mL) at RT. H₂SO₄ (200 μL) was added dropwise to this solution and the solution was heated at 65° C. for 3 d. The solution was cooled to RT and the solvent was removed under reduced pressure. H₂O (30 mL) was added to the residue and the solution was extracted with EtOAc (3×20 mL). The organic phases were combined, washed with 5% sodium bicarbonate solution (2×20 mL), and then with a solution of saturated brine (20 mL). After drying over MgSO₄, the solvent was filtered, and removed under reduced pressure to give compound 48, which was used in the rest of the synthesis without additional purification (57 mg, 76%). ¹H NMR (400 MHz, CDCl₃, δ): 8.33 (d, 1H, ⁴J 3.1, H⁵), 8.19 (d, 1H, ⁴J 3.1, H³), 4.02 (s, 3H, CH₃CO), 2.57 (s, 3H, py-CH₃); ¹³C NMR (100 MHz, CDCl₃, δ): 160.8 (COOMe), 152.7 (C⁶), 142.1 (C⁴), 140.5 (C²), 121.4 (C⁵), 119.3 (C³), 53.8 (OCH₃), 18.3 (py-CH₃); MS Calculated C₈H₉N₂O₅ 213.051. Found 213.050 [M+H]⁺.

Compound 49: trifluoroacetic anhydride (1.48 mL, 10.8 mmol) was added at RT to a solution of compound 48 (114 mg, 0.54 mmol) in CHCl₃ (10 mL). The mixture was heated at 60° C. for 5 h under inert atmosphere. After this time, the reaction was cooled to RT and then the solvent was removed under reduced pressure. EtOH (3 mL) and H₂O (3 mL) were added to the yellow oil and the solution was stirred at RT for 2 h. The solvents were removed under reduced pressure and the aqueous phase was extracted with CH₂Cl₂ (3×30 mL). The organic phases were combined, dried over MgSO₄ and evaporated under reduced pressure. The residue was purified by silica column chromatography using a Hexane/EtOAc solvent gradient, 70/30 to 50/50 to give compound 49 (74 mg, 65%). R_{f}(CH₂Cl₂/MeOH, 95/5)=0.67; ¹H NMR (400 MHz, CDCl₃, δ): 8.68 (d, 1H, ⁴J 2.1, H³), 8.37 (d, 1H, ⁴J 2.1, H⁵), 5.06 (s, 2H, CH₂OH), 4.06 (s, 3H, CH₃CO); ¹³C NMR (100 MHz, CDCl₃, δ): 164.3 (COOMe), 163.6 (C⁶), 155.3 (C⁴), 149.7 (C²), 116.4 (C⁵), 116.3 (C³), 64.5 (CH₂OH), 29.5 (CO₂CH₃).

Compound 50a: NaH (17 mg, 0.708 mmol) and ethyl thioglycolate (35 μL, 0.320 mmol) were added under inert atmosphere and at RT to a solution of compound 49 (21.6 mg, 0.102 mmol) in anhydrous DMF (1 mL). The mixture was stirred at RT for 2 h under inert atmosphere. The solvent was then removed under reduced pressure and MeOH (5 mL) and H₂SO₄ (200 μL) were added to the yellow oil. The solution was heated at 65° C. for 72 h under argon. The solvent was removed under reduced pressure and H₂O (10 mL) was added to the residue, and the aqueous solution was extracted with EtOAc (3×20 mL). The organic phases were combined and dried over MgSO₄, filtered and concentrated under reduced pressure. The residue was purified by silica column chromatography using as eluent CH₂Cl₂-MeOH, 98/2 to give compound 50a (8.2 mg, 25%). R_{f} (DCM/MeOH, 95/5)=0.35; ¹H NMR (400 MHz, CDCl₃, δ): 7.88 (d, 1H, ⁴J 1.9, H⁵), 7.63 (d, 1H, ⁴J 1.9, H³), 4.69 (s, 2H, CH₂OH), 4.02 (s, 2H, CH₂S), 3.96 (s, 3H, CH₃CO), 3.76 (s, 3H, CH₃CO); ¹³C NMR (100 MHz, CDCl₃, δ): 170.7 (COOMe), 166.4 (COOMe), 163.3 (C²), 152.3 (C⁴), 147.8 (C⁶), 121.2 (C⁵), 121.1 (C³), 65.1 (CH₂OH), 53.3 (CO₂CH₃), 48.5 (CO₂CH₃), 33.6 (SCH₂).

Compound 50b: compound 50b was prepared according to the same procedure as that used for the synthesis of 50a.

Compound 51a: TEA (12.5 μL, 0.09 mmol) and MsCl (3.5 μL, 0.045 mmol) were added to a solution of compound 50a (8.2 mg, 0.03 mmol) in anhydrous THF (2 mL). This solution was stirred at RT for 3.5 h. After this time, the solvent was removed under reduced pressure and the residue was dissolved in CH₂Cl₂ (20 mL). The organic phase was washed with H₂O (3×10 mL), dried over MgSO₄, filtered and the solvent was removed under reduced pressure to give compound 51a quantitatively. R_{f}(DCM/MeOH, 95/5)=0.8; ¹H NMR (400 MHz, CDCl₃, δ): 7.95 (d, 1H, ⁴J 1.5, H⁵), 7.52 (d, 1H, ⁴J 1.5, H³), 5.35 (s, 2H, CH₂OMs), 3.98 (s, 2H, CH₂S), 3.82 (s, 2H, COCH₃), 3.77 (s, 2H, COCH₃), 3.14 (s, 3H, SCH₃).

Compound 51b: compound 51b was prepared according to the same procedure as that used for the synthesis of 51a.

Compounds 52a-b: compounds 52a-b were prepared according to the same procedures as those used for the synthesis of 14b and 14c, respectively.

Compounds 53a-b: compounds 53a-b were prepared according to the same procedure as that used for the synthesis of 46.

Compounds 54a-b: compounds 54a-b were prepared according to the same procedure as that used for the synthesis of 49.

Compounds 55a-d: compounds 55a-d were prepared according to the same procedure as that used for the synthesis of 50a.

Compounds 56a-d: compounds 56a-d were prepared according to the same procedure as that used for the synthesis of 51a.

Compound 57: this compound is available commercially.

Compound 58: imidazole (6.49 g, 94.4 mmol) and then TBDMSCl (9.78 g, 62.9 mmol) were added to a solution of 3,5-dimethoxyphenol (10 g, 62.9 mmol) in anhydrous DMF (145 mL). The reaction mixture was stirred overnight at RT. H₂O (50 mL) was added to this solution and then the solution was extracted with EtOAc (2×30 mL). The organic phases were combined, washed with a solution of brine (20 mL), dried over MgSO₄, filtered and concentrated under reduced pressure. The crude product was purified by silica column chromatography using a Cyclohexane-EtOAc solvent gradient of 0/90-85/15 in increments of 5% to give compound 58 (15.8 g, 94%) in the form of colorless oil. HPLC—Rt=4.19 min—[M+H]⁺, m/z 270.3—¹H NMR (300 MHz, CDCl₃) δ: 6.11 (s, 1H, para), 6.03 (s, 2H, ortho), 3.75 (s, 6H, OMe), 0.98 (s, 9H, Si-tert-Bu), 0.21 (s, 6H, Si-Me)

Compound 59: n-BuLi 2.5 M in hexane (26.3 mL, 65.8 mmol) was added dropwise at −78° C. under argon to a solution of compound 58 (15.8 g, 58.9 mmol) in anhydrous THF (130 mL). The reaction mixture was stirred at RT for 5 h and then cooled to −78° C. A solution of 2-isopropoxy-4,4,5,5-tetramethyl-1,3,2-dioxaborolane (14.4 mL, 70.6 mmol) in anhydrous THF (32 mL) was added dropwise to this solution. The reaction mixture was stirred at RT for 3 h and was then poured into a crushed ice-H₂O mixture (400 mL). The aqueous phase was extracted with EtOAc (2×50 mL). The organic phases were combined, washed with a solution of brine (20 mL), dried over MgSO₄, filtered and concentrated under reduced pressure. MeOH (9 mL) was added to the residue and the solution was cooled to 4° C. overnight. After this time, a white solid had crystallized. The crystals were collected by filtration and dried to give compound 59 (8.91 g, 38%) in the form of a white solid. HPLC—Rt=3.80 min—[M+H]⁺, m/z 395.3—¹H NMR (300 MHz, CDCl₃) δ: 5.97 (s, 2H, ortho), 3.7 (s, 6H, OMe), 1.36 (s, 12H, B(O—C-diMe)), 0.96 (s, 9H, Si-tert-Bu), 0.17 (s, 6H, Si-Me); ¹³C NMR (300 MHz, CDCl₃) δ: 164.6; 159.4; 96.8; 83.8; 55.9; 26.1; 25.0; 18.6, −4.0;

Compounds 60a and 61a: in a 50-mL Schlenk flask, compound 14a (440 mg, 1.5 mmol) was dissolved in a mixture of acetone (2 mL) and H₂O (2.5 mL) to give a colorless solution. Compound 59 (710 mg, 1.8 mmol), K₂CO₃ (518 mg, 3.75 mmol), and Pd(dba)₂ (1.725 mg, 3.00 μmol) in solution in acetone (0.5 mL) were added to the reaction mixture in one go. The reaction mixture was stirred at 65° C. for 4 h. The progress of the reaction was monitored by UPLC-MS (Gradient A). After this time, the reaction was complete, containing a mixture of compounds 60a and 61a. The reaction mixture was concentrated under reduced pressure and was used in the rest of the synthesis without additional purification. HPLC gradient A—Rt=1.44 min—[M+H]⁺, m/z 304.6

Compound 61a: in a 250-mL flask, the mixture of compounds 60a and 61a (458 mg, 1.5 mmol) was dissolved in MeOH (100 mL) to give a yellow solution. H₂SO₄ (0.416 mL, 4.50 mmol) was added to the reaction mixture in one go. The reaction was stirred under reflux for 7 d. The progress of the reaction was monitored by UPLC-MS (gradient A). After this time, the reaction was partial (90%). The reaction mixture was concentrated under reduced pressure and was then purified directly by preparative HPLC (gradient G) to give compound 61a (385 mg, 1.21 mmol, 80%) in the form of yellow powder. mp=169.7-174.1° C.—HPLC gradient A—Rt=2.07 min—[M+H]⁺, m/z 320.3—HRMS (ESI+) calculated for C₁₆H₁₈NO₆⁺[M+H]⁺, m/z 320.1129, found: 320.1127—¹H NMR (400 MHz, MeOD₄) δ: 8.22 (s, 1H, Py H³), 8.02 (s, 1H, Py H⁵), 6.25 (s, 2H, ortho), 4.89 (s, 2H, Py-CH₂—OH), 4.05 (s, 3H, Py-COOMe), 3.77 (s, 6H, OMe); ¹³C NMR (100 MHz, MeOD₄) δ: 162.95; 161.64; 158.75; 158.68; 150.91; 141.38; 127.81; 127.22; 105.67; 91.95; 61.70; 54.89; 52.54.

Compound 62a: in a 250-mL flask, compound 61a (354 mg, 1.11 mmol) was dissolved in anhydrous MeCN (100 mL) to give a yellow solution. K₂CO₃ (460 mg, 3.33 mmol), KI (27.6 mg, 0.166 mmol) and then methyl bromoacetate (0.162 mL, 1.66 mmol) were added to the reaction mixture in one go. The reaction was stirred at 65° C. overnight. The progress of the reaction was monitored by UPLC-MS (gradient A). After this time, the reaction was complete. The reaction mixture was concentrated under reduced pressure, diluted in DCM (50 mL) and then filtered and finally purified by silica column chromatography using EtOAc as eluent to give compound 62a (261 mg, 60%) in the form of white powder. mp=151.1-154.4° C.—HPLC gradient A—Rt=2.58 min—[M+H]⁺, m/z 393.1—Rf=0.36 (silica, ethyl acetate)—HRMS (ESI+) calculated for C₁₉H₂₂NO₈⁺ [M+H]⁺, m/z 392.1340, found: 392.1340—¹H NMR (400 MHz, CDCl₃) δ: 8.03 (s, 1H, Py H³), 7.48 (s, 1H, Py H⁵), 6.24 (s, 2H, ortho), 4.87 (s, 2H, Py-CH₂—OH), 4.71 (s, 2H, O-CH₂—COOMe), 3.99 (s, 3H, Py-COOMe), 3.86 (s, 3H, O—CH₂—COOMe), 3.73 (s, 6H, OMe); ¹³C NMR (100 MHz, CDCl₃) δ: 169.03; 166.03; 159.85; 159.18; 158.20; 146.32; 144.61; 127.05; 126.72; 109.65; 91.48; 65.38; 64.71; 55.89; 52.73; 52.46.

Compound 63a: in a 100-mL flask, compound 62a (224 mg, 0.572 mmol) was dissolved in anhydrous THF (30 mL) to give a colorless solution. The reaction mixture was placed in an ice bath and then MsCl (45 μL, 0.572 mmol) was added in one go. At the end of addition, the ice bath was removed and the reaction was stirred for 15 min. The progress of the reaction was monitored by UPLC-MS (gradient A). After this time, the reaction was complete. The reaction mixture was concentrated under reduced pressure, diluted in DCM (50 mL) and washed with water (2×40 mL). The organic phase was dried over MgSO₄, filtered and evaporated to dryness in a rotary evaporator. The crude product was purified by silica column chromatography using a DCM/MeOH solvent gradient from 100/0 to 95/5 to give compound 63a (264 mg, 98%) in the form of white powder. mp=141.7-144.1° C.—HPLC gradient A—Rt=2.96 min—[M+H]⁺, m/z 470.6—Rf=0.48 (silica, dichloromethane—methanol 96:4)—HRMS (ESI+) calculated for C₂₀H₂₄NO₁₀S⁺ [M+H]⁺, m/z 470.1115, found: 470.1113—¹H NMR (400 MHz, CDCl₃) δ: 8.13 (s, 1H, Py H³), 7.67 (s, 1H, Py H⁵), 6.24 (s, 2H, ortho), 5.46 (s, 2H, Py-CH₂—OH), 4.72 (s, 2H, O-CH₂—COOMe), 4.00 (s, 3H, Py-COOMe), 3.86 (s, 3H, O—CH₂—COOMe), 3.73 (s, 6H, OMe), 3.14 (s, 3H, OMs); ¹³C NMR (100 MHz, CDCl₃) δ: 168.98; 165.73; 160.09; 158.20; 153.24; 147.15; 145.30; 128.25; 128.10; 109.08; 91.49; 71.66; 65.37; 55.90; 52.94; 52.48; 38.17.

Compound 77a: in a 50-mL Schlenk flask, compound 1b (41 mg, 0.159 mmol) was dissolved in anhydrous THF (5 mL) to give a colorless solution. Compound 63a (223 mg, 0.476 mmol) in solution in anhydrous MeCN (10 mL) and then K₂CO₃ (88 mg, 0.635 mmol) were added to the reaction mixture in one go. The reaction was stirred at 85° C. overnight. The progress of the reaction was monitored by UPLC-MS (gradient A). After this time, the reaction was complete. The reaction mixture was purified directly by preparative HPLC (gradient H) to give compound 77a (106 mg, 48%) in the form of white powder. HPLC gradient A—Rt=3.64 min—[M+H]⁺, m/z 1379.9—HRMS (ESI+) calculated for C₂₀H₂₄N₁₀S⁺ [M+2H]²⁺, m/z 689.7843, found: 689.7842.

Compound Eu-79a: in a 50-mL flask, compound 77a (53 mg, 38.5 μmol) was dissolved in MeCN (1 mL) and water (5 mL) to give a colorless solution. LiOH (0.941 mg, 38.5 μmol) was added to the reaction mixture in one go. The reaction was stirred at room temperature for 30 min. The progress of the reaction was monitored by UPLC-MS (gradient A). After this time, the deprotection was complete (compound 78a). The pH of the reaction mixture was adjusted to 7 with 1M HCl. Europium chloride hexahydrate (21 mg, 57.8 μmol) was added to the reaction mixture in one go. The reaction was stirred at RT overnight, after this time the reaction was complete. The reaction mixture was purified directly by preparative HPLC (gradient D) to give compound 79a (49 mg, 88%) in the form of white powder. HPLC gradient A—Rt=2.63 min—[M-2H]⁺, m/z 1445.4—HRMS (ESI+) calculated for C₆₃H₇₀N₇O₂₃Eu²⁺ [M−H]²⁺, m/z 722.6868, found: 722.6868.

Compound Eu-80a-E₂: in a 25-mL flask, compound 79a (49 mg, 34 μmol) was dissolved in anhydrous DMSO (1.5 mL) to give a colorless solution. 3-Amino-1-propanesulfonic acid (29 mg, 204 μmol), DIPEA (36 μL, 204 μmol) and then HATU (53 mg, 136 μmol) were added to the reaction mixture in one go. The reaction was stirred at RT for 15 min. The progress of the reaction was monitored by UPLC-MS (gradient C). After this time, the reaction was complete. The reaction mixture was purified directly by preparative HPLC (gradient D) to give compound Eu-80a-E₂ (19 mg, 10.2 μmol, 30%) in the form of white powder. HPLC gradient C—Rt=1.92 min—[M-2H]⁺, m/z 1808.4—HRMS (ESI+) calculated for C₇₂H₉₁N₁₀O₂₉S₃Eu²⁺[M−H]²⁺, m/z 904.2162, found: 904.2166.

Compound Eu-81a-E₂: in a 25-mL flask, compound Eu-80a-E₂ (18.32 mg, 10.14 μmol) was dissolved in TFA (400 μL) to give a yellow solution. The reaction was stirred at RT for 30 min. The progress of the reaction was monitored by UPLC-MS (gradient C). After this time, the reaction was complete. The reaction mixture was evaporated in a rotary evaporator and then purified by preparative HPLC (gradient E) to give compound Eu-81a-E₂ (7.89 μmol, 78%) in the form of white powder. HPLC gradient C—Rt=1.48 min—[M-2H]⁺, m/z 1709—HRMS (ESI+) calculated for C₆₇H₈₃N₁₀O₂₇S₃Eu²⁺ [M−H]²⁺, m/z 854.1899, found: 854.1906.

Compound Tb-79a: in a 50-mL flask, compound 77a (53 mg, 38.5 μmol) was dissolved in MeCN (1 mL) and water (5 mL) to give a colorless solution. LiOH (0.941 mg, 38.5 μmol) was added to the reaction mixture in one go. The reaction was stirred at RT for 30 min. The progress of the reaction was monitored by UPLC-MS (gradient A). After this time, the deprotection was complete (compound 78a). The pH of the reaction mixture was adjusted to 7 with 1M HCl. Terbium chloride hexahydrate (22 mg, 57.8 μmol) was added to the reaction mixture in one go. The reaction was stirred at RT overnight; after this time, the reaction was complete. The reaction mixture was purified directly by preparative HPLC (gradient D) to give compound Tb-79a (19 mg, 12.8 μmol, 33%) in the form of white powder. HPLC gradient A—Rt=2.63 min—[M-2H]⁺, m/z 1451.7—HRMS (ESI+) calculated for C₆₃H₇₀N₇O₂₃Tb²⁺ [M−H]²⁺, m/z 725.6883, found: 725.6887.

Compound Tb-80a-E₂: in a 25-mL flask, compound Tb-79a (9.3 mg, 6.4 μmol) was dissolved in anhydrous DMSO (1 mL) to give a colorless solution. 3-Amino-1-propanesulfonic acid (5.5 mg, 38.4 μmol), DIPEA (4.5 μL, 25.6 μmol) and then HATU (10 mg, 25.6 μmol) were added to the reaction mixture in one go. The reaction was stirred at RT for 15 min. The progress of the reaction was monitored by UPLC-MS (gradient C), After this time, the reaction was complete. The reaction mixture was purified directly by preparative HPLC (gradient D) to give compound Tb-80a-E₂ (7.8 mg, 4.3 μmol, 67%) in the form of white powder. HPLC gradient C—Rt=1.89 min—[M-2H]⁺, m/z 1815.9—HRMS (ESI+) calculated for C₇₂H₉₁N₁₀O₂₉S₃Tb²⁺ [M−H]²⁺, m/z 907.2179, found: 907.2167.

Compound Tb-80a-E₄: in a 25-mL flask, compound Tb-79a (9.3 mg, 6.4 μmol) was dissolved in anhydrous DMSO (1 mL) to give a colorless solution. 2-N,N,N-Trimethylammonium-ethylamine (3.96 mg, 38.4 μmol), DIPEA (4.5 μL, 25.6 μmol) and then HATU (10 mg, 25.6 μmol) were added to the reaction mixture in one go. The reaction was stirred at RT for 15 min. The progress of the reaction was monitored by UPLC-MS (gradient C). After this time, the reaction was complete. The reaction mixture was purified directly by preparative HPLC (gradient D) to give compound Tb-80a-E₄ (6.1 mg, 3.6 μmol, 56%) in the form of white powder. HPLC gradient C—Rt=2.00 min—[M]³⁺, m/Z 569.3—HRMS (ESI+) calculated for C₇₈H₁₀₈N₁₃O₂₀Tb⁴⁺[M-2H]⁴⁺, m/z 426.4266, found: 426.4265.

Compound Tb-81a-E₂: in a 25-mL flask, compound Tb-80a-E2 (7.8 mg, 4.3 μmol) was dissolved in TFA (500 μL) to give a yellow solution. The reaction was stirred at RT for 30 min. The progress of the reaction was monitored by UPLC-MS (gradient C). After this time, the reaction was complete. The reaction mixture was evaporated in a rotary evaporator and then purified by preparative HPLC (gradient E) to give compound Tb-81a-E₂ (2.78 μmol, 64%) in the form of white powder. HPLC gradient C—Rt=1.45 min—[M-2H]⁺, m/z 1715.3—HRMS (ESI+) calculated for C₆₇H₈₃N₁₀O₂₇S₃Tb²⁺ [M−H]²⁺, m/z 857.1917, found: 857.1905.

Compound Tb-81a-E₄: in a 25-mL flask, compound Tb-80a-E₄ (6.1 mg, 3.6 μmol) was dissolved in TFA (200 μL) to give a yellow solution. The reaction was stirred at RT for 30 min. The progress of the reaction was monitored by UPLC-MS (gradient C). After this time, reaction was complete. The reaction mixture was evaporated in a rotary evaporator and then purified by preparative HPLC (gradient E) to give compound Tb-81a-E₄ (2.2 μmol, 62%) in the form of white powder. HPLC gradient C—Rt=1.57 min—[M]³⁺, m/z 535.9—HRMS (ESI+) calculated for C₇₃H₁₀₀N₁₃O₁₈Tb⁴⁺ [M-2H]⁴⁺, m/z 401.4135, found: 401.4125.

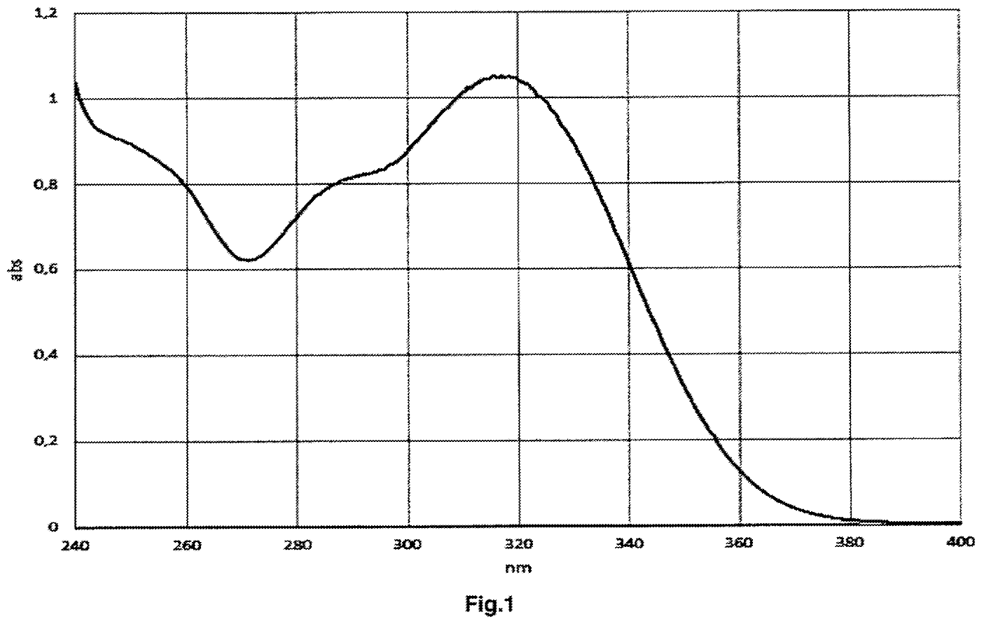

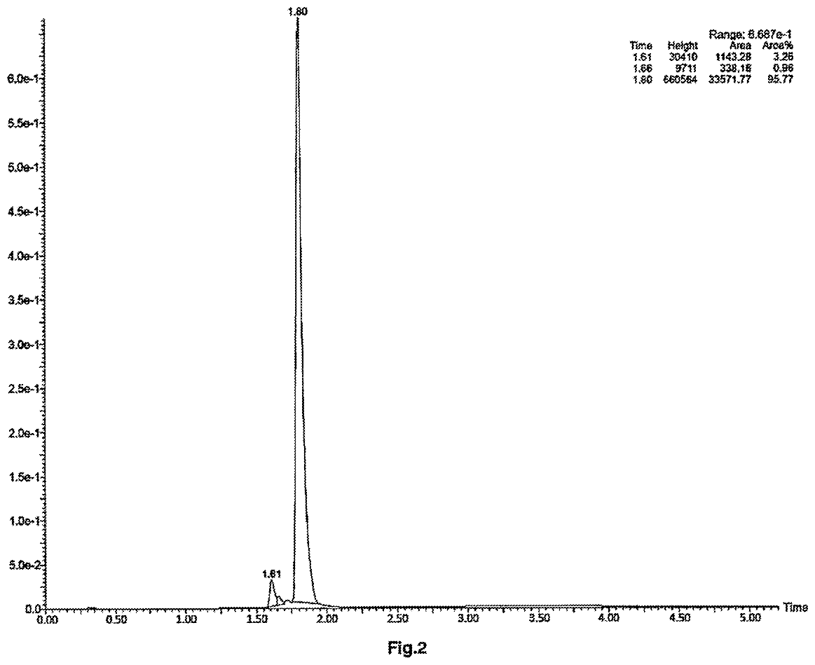

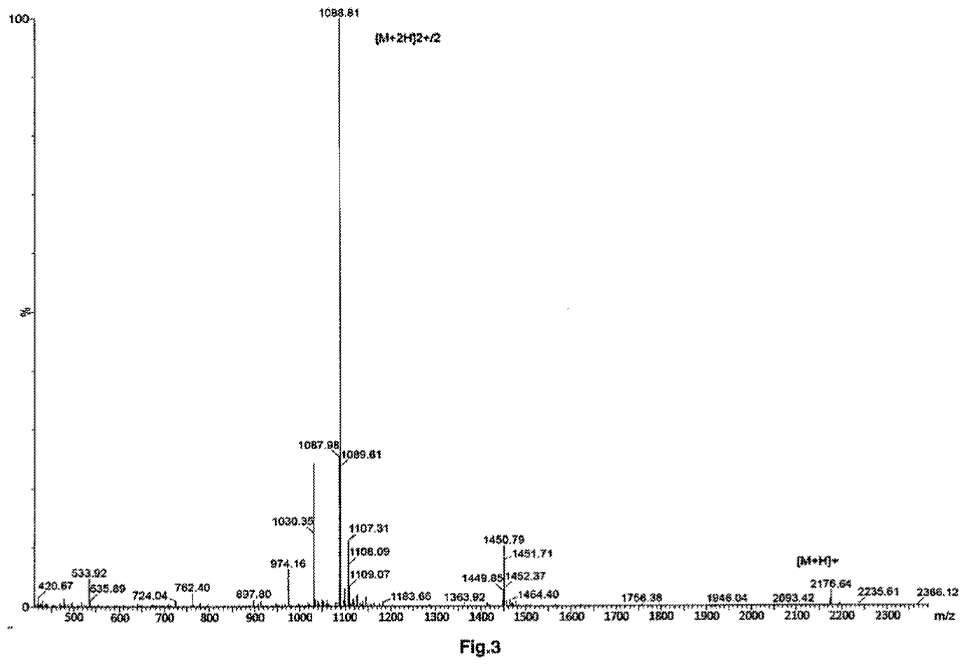

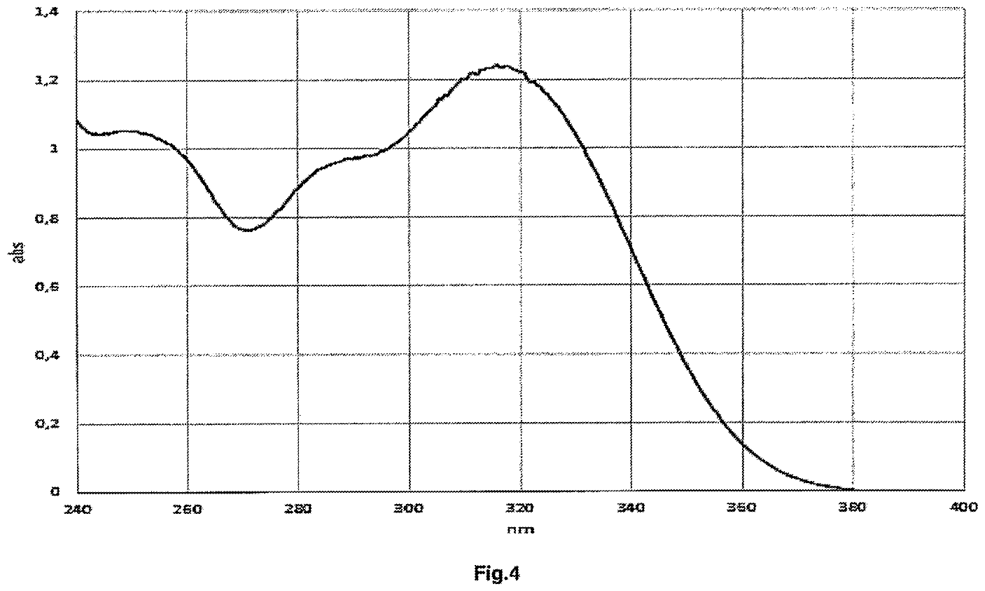

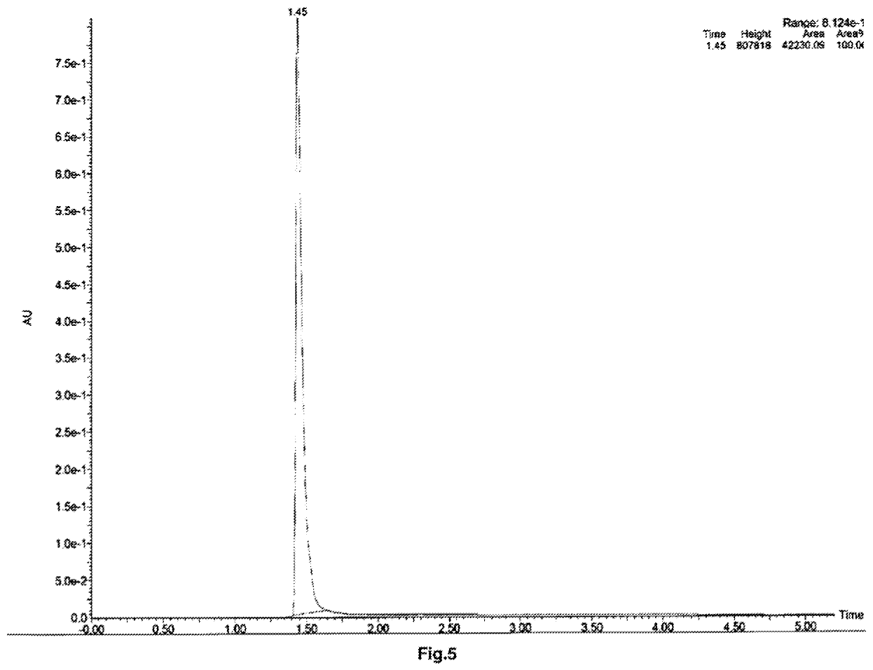

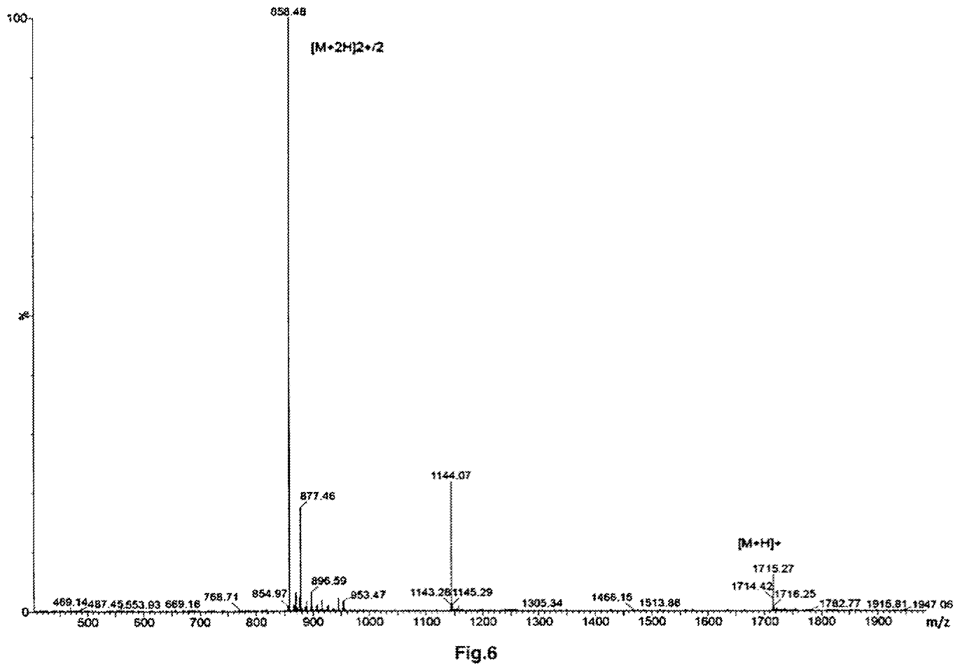

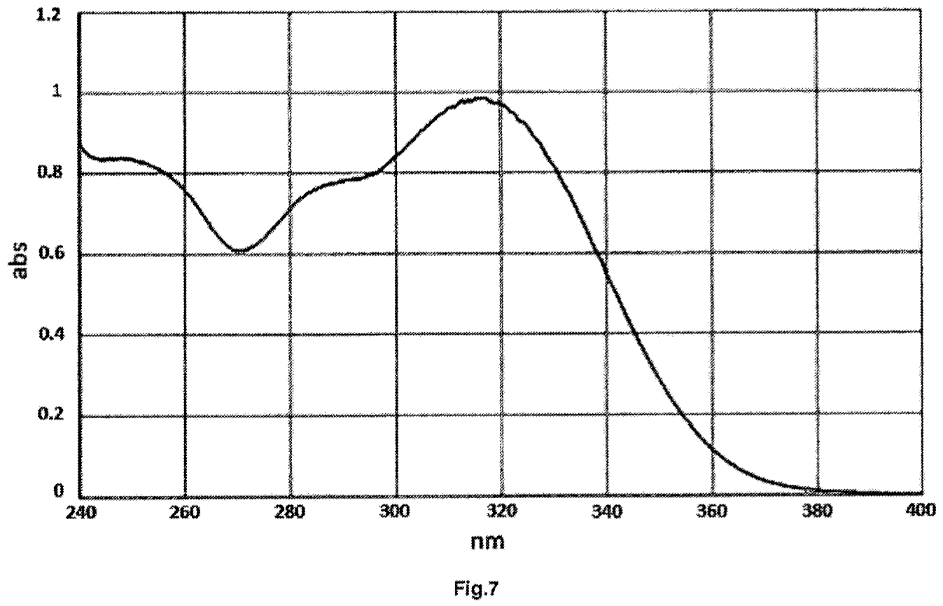

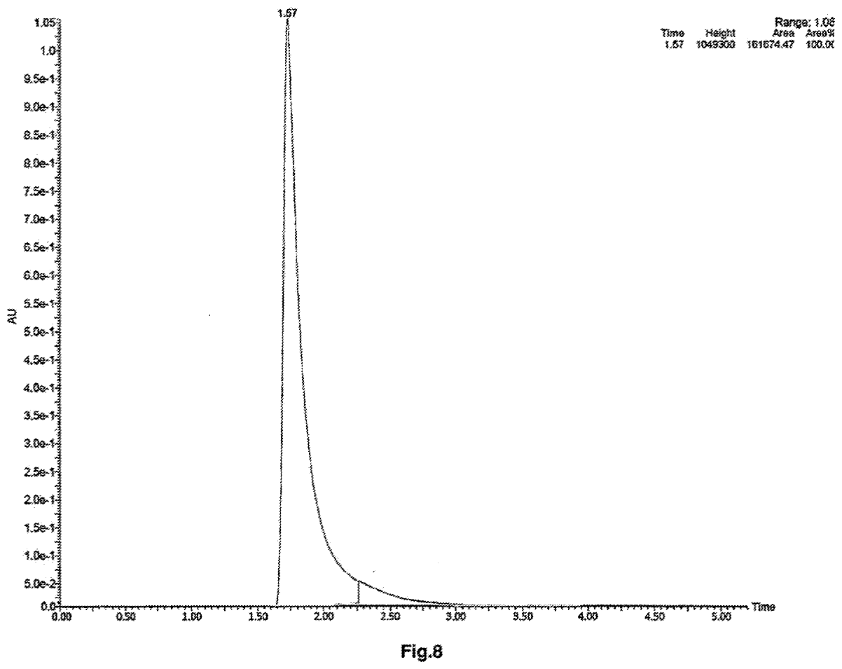

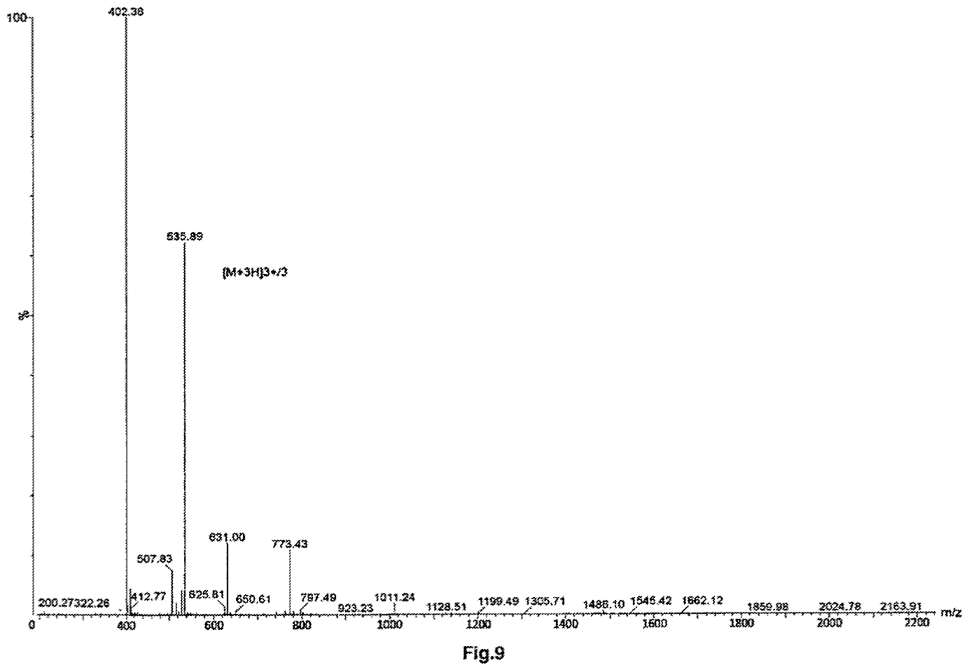

The UV spectrum, the chromatogram and the mass spectrum of the complex Eu-81a-E₂ are shown in FIGS. 1 to 3. The UV spectrum, the chromatogram and the mass spectrum of the complex Tb-81a-E₂ are shown in FIGS. 4 to 6. The UV spectrum, the chromatogram and the mass spectrum of the complex Tb-81a-E₄ are shown in FIGS. 7 to 9.

The properties of the complexes Eu-81a-E2, Tb81a-E4 and Tb-81a-E2, and of the complexes corresponding to the structures 82a and 82b, described in application WO 2005/058877, were determined. The photophysical properties of complexes 82a and Eu-81a-E2 are comparable. However, complex Eu-81a-E2 is very water-soluble whereas complex 82a has very poor solubility (see below). Regarding the terbium complexes, the photophysical properties of complexes 82b and Tb81a-E4 and Tb-81a-E2 are comparable, although there are small differences regarding the intensity and distribution of the lines of the emission spectrum. However, complexes Tb81a-E4 and Tb-81a-E2 are very water-soluble compared to the complex of the prior art 82b (see below).

The solubility of the various complexes was determined as follows. For each complex, three equimolar solutions of europium complex were prepared in methanol. The solvent was removed under reduced pressure and the solid that remained was dissolved and stirred for 2 min in a water/octanol mixture (2:1, 1:1, 1:2), (0.9 mL). After equilibration, an emission spectrum of each phase was recorded in methanol (50 μL of solution in 1 mL of methanol). For each mixture, the value of Log P was calculated using the following equation:

Log P=Log [C(octanol)/C(water)]

in which C(octanol) and C(water) represent the concentration of the complex tested in octanol and in water, respectively.

For the europium complexes, the band ΔJ=2 (605-635 nm) was used in the calculations, whereas for the terbium complexes, the band ΔJ=5 (520-565 nm) was chosen. The results are presented in the following table.

| Complex | LogP |
|---|---|
| 82a | 0.4 ± 0.2 |
| 82b | 0.5 ± 0.1 |
| Eu-81a-E2 | −2.7 ± 0.2 |
| Tb-81a-E2 | −2.8 ± 0.2 |
| Tb-81a-E4 | −2.3 ± 0.2 |

The Log P values of the complexes according to the invention are negative, which reflects perfect solubility in the aqueous buffers, in contrast to the compounds 82a and 82b, whose Log P values are slightly positive.

1. A complexing agent of formula (I): 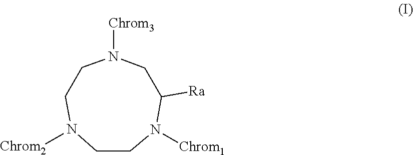
wherein:
Chrom₁, Chrom₂ and Chrom₃ each represent a group of formula (Ia) or (Ib): 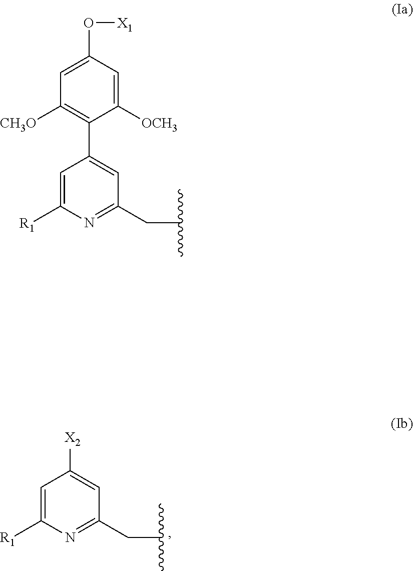
X₁ and X₂ each represent a group L₁-CO—R or L₂-G;
R is a group —OR₂ or —NH-E;
Ra is H or a group —(CH₂)ₗ-G;
R₁ is a group —CO₂H or —PO(OH)R₃;
R₂ is H or a (C₁-C₄)alkyl;
R₃ is a (C₁-C₄)alkyl; a phenyl optionally substituted with a group —SO₃⁻; or a benzyl;
L₁ is a direct bond; a group —(CH₂)ᵣ— optionally interrupted by at least one atom selected from the group consisting of an oxygen atom, a nitrogen atom and a sulfur atom; a —CH═CH— group; a —CH═CH—CH₂— group; a —CH₂—CH═CH— group; or a PEG group;
L₂ is a divalent linking group;
G is a reactive group;
E is a group —CH₂—(CH₂)ₛ—CH₂—SO₃⁻ or —N⁺Alk₁Alk₂Alk₃, or a sulfobetaine;
l is an integer in the range from 1 to 4;
r is an integer in the range from 1 to 6;
s is 0, 1 or 2;
Alk₁, Alk₂, Alk₃, which may be identical or different, represent a (C₁-C₆)alkyl;
it being understood that the compound of formula (I) comprises at least one group of formula (Ia) and at least one group L₁-CO—R.

2. The complexing agent as claimed in claim 1, wherein Chrom₁ represents a group of formula (Ia) in which X₁ is a group L₂-G; and Chrom₂ and Chrom₃ each represent a group of formula (Ib) in which X₂ is a group L₁-CO—R.

3. The complexing agent as claimed in claim 2, wherein Chrom₂ and Chrom₃ are identical.

4. The complexing agent as claimed in claim 1, wherein Chrom₁ and Chrom₂ each represent a group of formula (Ia) in which X₁ is a group L₁-CO—R; and Chrom₃ represents a group of formula (Ib) in which X₂ is a group L₂-G.

5. The complexing agent as claimed in claim 4, wherein Chrom₁ and Chrom₂ are identical.

6. The complexing agent as claimed in claim 2, wherein Ra is H.

7. The complexing agent as claimed in claim 1, wherein Chrom₁, Chrom₂ and Chrom₃ each represent a group of formula (Ia) in which X₁ is a group L₁-CO—R; and Ra is a group —(CH₂)ₗ-G.

8. The complexing agent as claimed in claim 8, wherein Chrom₁, Chrom₂ and Chrom₃ are identical.

9. The complexing agent as claimed in claim 1, wherein R₁ is a group —CO₂H or —P(O)(OH)R₃ in which R₃ is a (C₁-C₄)alkyl or a phenyl.

10. The complexing agent as claimed in claim 1, wherein L₁ is a direct bond; a group —(CH₂)ᵣ— optionally interrupted by at least one atom selected from an oxygen atom and a sulfur atom, and r=2 or 3; a —CH═CH— group; a —CH═CH—CH₂— group; or a —CH₂—CH═CH group.

11. The complexing agent as claimed in claim 1, wherein E is a group —CH₂—(CH₂)ₛ—CH₂—SO₃⁻ with s=0 or 1; —(CH₂)ₛ—N⁺Alk₁Alk₂Alk₃ with Alk₁, Alk₂ Alk₃, which may be identical or different, representing a (C₁-C₄)alkyl and s=0 or 1; or a group of formula: 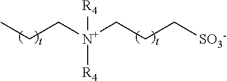
in which R₄ is a (C₁-C₄)alkyl and t is 1 or 2.

12. The complexing agent as claimed in claim 1, wherein L₂ is selected from:
a direct bond;
a linear or branched C₁-C₂₀ alkylene group, optionally containing one or more double or triple bonds;
a C₅-C₈ cycloalkylene group;
a C₆-C₁₄ arylene group;
said alkylene, cycloalkylene or arylene groups optionally containing one or more heteroatoms, or one or more carbamoyl or carboxamido group(s), and said alkylene, cycloalkylene or arylene groups optionally being substituted with 1 to 5 C₁-C₈ alkyl, C₆-C₁₄ aryl, sulfonate or oxo groups; and
a divalent group of formula: 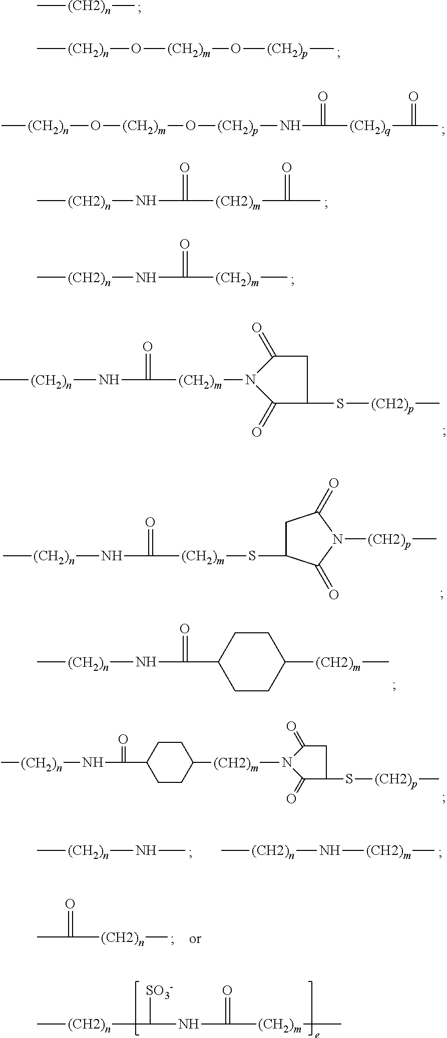
in which n, m, p, q are integers from 1 to 16 and e is an integer in the range from 1 to 6.

13. The complexing agent as claimed in claim 1, wherein the reactive group G is selected from the group consisting of: an acrylamide, an activated ester, an aldehyde, an alkyl halide, an anhydride, an aniline, an azide, an aziridine, a carboxylic acid, a diazoalkane, a haloacetamide, a halotriazine, a hydrazine, an imido ester, an isocyanate, an isothiocyanate, a maleimide, a sulfonyl halide, a thiol, a ketone, an amine which is optionally activated, an acid halide, a succinimidyl ester, a hydroxysuccinimidyl ester, a hydroxysulfosuccinimidyl ester, an azidonitrophenyl, an azidophenyl, a glyoxal, a triazine, and an acetylene group

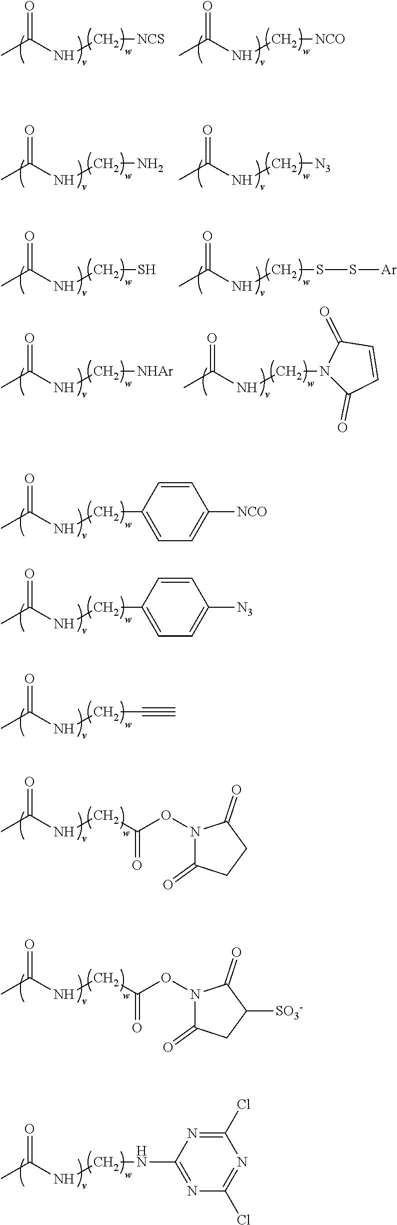

14. The complexing agent as claimed in claim 1, wherein the group -L₂-G consists of (i) a reactive group G selected from the group consisting of: a carboxylic acid, an amine, a succinimidyl ester, a haloacetamide, a hydrazine, an isothiocyanate, and a maleimide group, and ii a spacer arm L₂ consisting of an alkylene chain comprising from 1 to 5 carbon atoms or a group selected from the groups of formula: 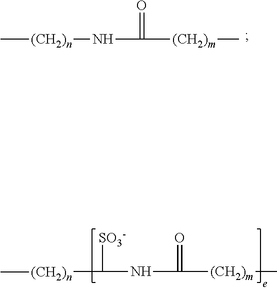
where n, m are integers ranging from 1 to 16 and e is an integer in the range from 1 to 6, the group G being bound to one or other end of these divalent groups.

15. A lanthanide complex comprising a complexing agent as claimed in claim 1 and a lanthanide.

16. The lanthanide complex as claimed in claim 15, wherein the lanthanide is selected from the group consisting of: Eu^{3+,} Tb^{3+,} and Sm³⁺.

17. A fluorescent conjugate of (i) a lanthanide complex as claimed in claim 15 and (ii) a molecule of interest, wherein said molecule of interest is covalently bonded to said lanthanide complex.

18. The complexing agent as claimed in claim 13, wherein the reactive group G is a group of formula: 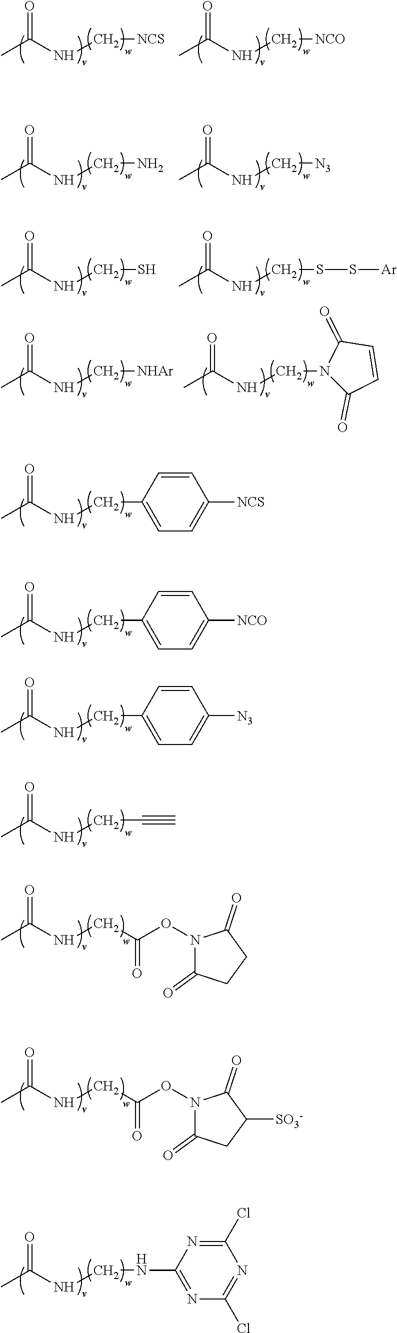
in which w varies from 0 to 8 and v is equal to 0 or 1, and Ar is a saturated or unsaturated 5- or 6-membered heterocycle, comprising 1 to 3 heteroatoms, optionally substituted with a halogen atom.

19. The lanthanide complex as claimed in claim 16, wherein the lanthanide is Tb³⁺.

20. The conjugate as claimed in claim 17, wherein the lanthanide of the lanthanide complex is selected from the group consisting of: Eu³⁺, Tb³⁺, and Sm³⁺.
